(19) United States
(12) Patent Application Publication
(10) Pub. No.: US 2005/0260268 A1
(43) Pub. Date: Nov. 24, 2005

(54) Methods and dosage forms for controlled delivery of alprazolam

(76) Inventors: Nishit B. Modi, Sunnyvale, CA (US); Suneel K. Gupta, Sunnyvale, CA (US); Nipun Davar, Fremont, CA (US); Sonya M. Seroff, San Jose, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/949,900

(22) Filed: Sep. 24, 2004

Related U.S. Application Data
(60) Provisional application No. 60/506,544, filed on Sep. 26, 2003.
(60) Provisional application No. 60/527,434, filed on Dec. 5, 2003.

(51) Int. Cl. A61K 9/24

(52) U.S. Cl. 424/473

(57) ABSTRACT

A dosage form for delivery of alprazolam is described. The sustained release dosage form provides via once-a-day dosing a therapeutically effective average steady-state plasma alprazolam concentration, where the maximum attained plasma concentration is achieved more than about 14 hours after administration. The slow, sustained release reduces side effects such as sedation and abuse potential.

### CROSS-REFERENCE TO RELATED APPLICATIONS

This application claims benefit of U.S. provisional patent application No. 60/506,544, filed Sep. 26, 2003, and to U.S. provisional patent application No. 60/527,434, filed Dec. 5, 2003. Both documents are incorporated herein by reference in their entirety.

### FIELD OF THE INVENTION

This invention relates to a dosage form for delivery of alprazolam. Alprazolam is released from the dosage form in a fashion that permits once daily dosing. The invention also relates to methods of treating conditions responsive to alprazolam.

### BACKGROUND OF THE INVENTION

Alprazolam is prescribed for the management of generalized anxiety disorders, for the treatment of panic disorder, and for short-term relief of symptoms associated with anxiety. The drug can be administered in a conventional dosage form, such as a nonrate-controlling, dose-dumping immediate release tablet, or by a dose-dumping capsule. When administered in a conventional, platform multiple, repetitive doses throughout the day are recommended (Evans, R. L. Psychiatric Annals, Supplement to October 1993 Issue, 8-13 (1993)). Alprazolam is also administered on a twice-a-day basis with a controlled release bead system identified by the tradename Xanax XR® (Evans, R. L. Id.). Despite the label claim, when administered in the controlled release bead system of Xanax XR® clinical practice suggests that twice-a-day dosing is needed, consistent with the twice-a-day dose labeling in Europe.

Alprazalom when administered from the controlled release bead system of Xanax XR® yields an initial maximum alprazolam blood concentration about ten hours after dosing, with a descending blood concentration thereafter, requiring a second dose to maintain therapeutic blood levels (Evans, R. L. Id.). This peak and trough occurs twice during a 24-hour period due to the twice-a-day dosing regimen. The peak and trough phenomena produced by known dosage forms is a drawback, as such a delivery profile results in a peak concentration that is higher than therapeutically necessary and a trough concentration that is lower than necessary to provide a therapeutic benefit. Moreover, the peak and trough delivery pattern provided by known dosage forms results in undesirable effects, such as sedation from over medicating at the peak concentration and reduced therapeutic benefit as the concentration falls below efficacious levels at the trough.

Such peaks and troughs are particularly undesirable for alprazolam, which demonstrates a steep dose response curve for increasing doses of alprazolam relative to measures that indicate sedation, memory impairment, and abuse potential. The issue of sedation and impaired motor impairment in the elderly is a particular concern with alprazolam.

Dosage forms for the controlled release of pharmaceutical agents are known in the art. For example, devices in which a drug composition is delivered as a slurry, suspension, or solution from a small exit orifice by the action of an expandable layer are described in U.S. Pat. Nos. 5,633,011; 5,190,765; 5,252,338; 5,620,705; 4,931,285; 5,006,346; 5,024,842; and 5,160,743. Typical devices include an expandable push layer and a drug layer surrounded by a semipermeable membrane. In certain instances, the drug layer is provided with a subcoat to delay release of the drug composition to the environment of use or to form an annealed coating in conjunction with the semipermeable membrane. Devices in which a drug composition is delivered in a dry state from a large exit orifice by the action of an expandable layer are described in U.S. Pat. Nos. 4,892,778, 4,915,949 and 4,940,465.

There remains a need for an effective dosage form that provides a controlled release of alprazolam over a period of time sufficient to permit a once per day dosing to provide effective therapy and a reduction in undesirable side effects associated with alprazolam dosing.

### SUMMARY OF THE INVENTION

In one aspect, the present invention provides a dosage form for the delivery of alprazolam. The dosage form of the present invention is preferably designed to be a once-a-day dosage form and to provide continuous management of central nervous system disorders through delivery of therapeutically effective amounts of alprazolam over 24 hours.

In one aspect, the invention includes a dosage form comprising a dose of alprazolam, the dosage form having a dissolution rate where between 25% and 60% of the dose is released 10 hours after exposure to an aqueous environment. In one embodiment, the dosage form provides a dissolution rate where between 35% and 55% of the dose is released 10 hours after exposure to an aqueous environment.

In another embodiment, the dosage is effective to provide a dissolution rate where less than 20% of the dose is released 2 hours after exposure to an aqueous environment.

In yet another embodiment, the dosage form is effective to provide a dissolution rate where between 30% and 80% of the dose is released 12 hours after exposure to an aqueous environment. In an alternative embodiment, the dosage form provides a dissolution rate where between 40% and 70% of the dose is released 10 hours after exposure to an aqueous environment.

In one embodiment, the dosage form is an osmotic dosage form. Such an osmotic dosage form, in one embodiment, is comprised of (i) a push layer; (ii) drug layer comprising alprazolam; (iii) a semipermeable wall provided around the push layer and the drug layer; and (iv) an exit. Alternatively, the osmotic dosage form is comprised of (i) a semipermeable wall provided around an osmotic formulation comprising an alprazolam formulation, an osmagent, and an osmopolymer; and (ii) an exit.

The dosage form, in one embodiment, provides a total daily dose of between 0.25-25 mg. In another embodiment, the dosage form provides a total daily dose of between 0.5 and 6 mg.

In another aspect, the invention provides a dosage form, comprising, a dose of alprazolam, where the dosage form is effective to provide an in vitro release profile where (i) less than 20% of the dose is released 2 hours after exposure to an aqueous environment; (ii) between 25%and 65% of the dose is released 10 hours after exposure to an aqueous environment; and (iii) greater than 85% of the dose is released 24 hours after exposure to an aqueous environment.

In another aspect, a dosage form for delivery of alprazolam is provided, where the dosage form is configured to release at least about 10%, more preferably 15%, of the dose 16 hours, more preferably 14 hours, after exposure to an aqueous environment.

In another aspect, the invention provides a dosage form for delivery of alprazolam, the dosage form comprising a dose of alprazolam and being configured to release at least about 25% of the dose 12 hours after exposure to an aqueous environment. Alternatively, the dosage form is designed to release at least about 30% of the dose 12 hours after exposure to an aqueous environment.

In another aspect, the invention includes a dosage form comprising alprazolam, where the dosage form provides a cumulative amount of drug released in vivo of between 25% and 60% at 10 hours, alternatively 12 hours, after oral delivery.

In one embodiment, the dosage form provides an in vivo release profile where between 35% and 55% of the dose is released 10 hours after exposure to an aqueous environment, i.e., after oral ingestion. In another embodiment, the dosage form is effective to provide a release profile where less than 20% of the dose is released 2 hours after exposure to an aqueous environment. In yet another embodiment, the dosage form is effective to provide a cumulative amount of drug released in vivo of between 30% and 80% of the total dose 12 hours after exposure to an aqueous environment. In an alternative embodiment, the dosage form provides a release profile where between 40% and 70% of the dose is released 10 hours after exposure to an aqueous environment.

In another aspect, the invention provides a dosage form comprising alprazolam, wherein the dosage form provides a maximum attained alprazolam plasma concentration (Cₘₐₓ) more than about 14 hours after administration. In one embodiment, the Cₘₐₓ occurs more than 16 hours after administration.

In another aspect, the invention provides a dosage form comprising alprazolam, where the dosage form provides a dose-normalized (normalized to 1 mg dose) area under the curve of less than about 110 ng·hr/mL·mg. In one embodiment, the dose-normalized area under the curve is greater than 70 ng·hr/mL·mg and less then about 110 ng·hr/mL·mg.

In other aspects, the invention contemplates methods of administering alprazolam to a human subject by administering the dosage form described above.

The invention also contemplates a method of treating a condition responsive to alprazolam, by administering a dosage form effective to provide a dose-normalized area under the curve of less than about 110 ng·hr/mL·mg.

The invention also contemplates a method of reducing side-effects associated with oral delivery of alprazolam when administered from an immediate release dosage formulation. In one embodiment, sedation caused by alprazolam is reduced by at least two-fold relative to the immediate release dosage form, when sedation is measured using a conventional test, such as those described hereinbelow.

These and other objects and features of the invention will be more fully appreciated when the following detailed description of the invention is read in conjunction with the accompanying drawings.

### BRIEF DESCRIPTION OF THE FIGURES

The following figures are set forth to illustrate various embodiments of the invention. Figures that include representations of one or more dosage forms are provided for illustrative purposes, are not necessarily drawn to scale, and are not meant to limit the scope of the present invention.

FIGS. 1A-1B show the average amount of drug released per hour, in mg/hr, (FIG. 1A) and the normalized cumulative amount of drug released as a percent of total drug amount (FIG. 1B), as a function of time, in hours, for an exemplary dosage form;

FIGS. 2A-2C show the in vitro dissolution rates into a release medium simulating artificial gastric fluid (AGF, FIG. 2A, FIG. 2C squares) and artificial intestinal fluid (AIF, FIG. 2B, FIG. 2C triangles) of alprazolam from a dosage form comprising 2 mg of alprazolam,

FIGS. 3A-3C are plots showing the in vitro amount of alprazolam released (reported as percent total dose released at each time point) from dosage forms comprising 0.5 mg (diamonds) or 2 mg (squares) alprazolam, as a function of time, in hours, with the amount released at each time plus and minus 15% (FIG. 3A), 12% (FIG. 3B) and 10% (FIG. 3C) shown in dashed lines;

FIGS. 4A-4B are schematic illustrations of exemplary dosage forms for delivery of alprazolam, FIG. 4A showing a dosage form in cutaway view and FIG. 4B showing a bilayer dosage form in cross-sectional view;

FIGS. 5A-5C show release profiles of alprazolam as a function of time from a dosage form prepared without sodium chloride (FIG. 5A; squares FIG. 5C) and with 20% sodium chloride (FIG. 5B, triangles FIG. 5C) in the drug layer of the dosage form;

FIGS. 6A-6C show dissolution rate profiles of alprazolam as a function of time from dosage forms prepared with 20% sodium chloride (FIG. 6A; squares FIG. 6C) and with 30% sodium chloride (FIG. 6B, triangles FIG. 6C) in the drug layer of the dosage form;

FIG. 7 is a plot of alprazolam plasma concentration, in ng/mL, as a function of time after a single dose of a SLOW dosage form at dosages of 1 mg (diamonds), 2×1 mg (squares), and 3×1 mg (triangles), and after a single dose of comparative controls of (i) FAST controlled release (2×1 mg, x symbols) and (ii) immediate release (2 mg; * symbols);

FIG. 8 is a bar graph showing the monetary value, in dollars, attributed by test subjects to receive an additional dose of drug from each of the test formulations in Treatments A-F, corresponding respectively to placebo (Treatment A), SLOW dosage form at dosages of 1 mg (Treatment B), 2×1 mg (Treatment C), and 3×1 mg (Treatment D), FAST controlled release (2×1 mg, Treatment E), and immediate release (2 mg, Treatment F);

FIG. 9 is an assessment of potential for psychomotor impairment resulting from each dosage form using a manual tracking evaluation of mean percent of time over-the-road as a function of time, in hours, after administration of a placebo (diamonds), SLOW dosage forms (1 mg, squares; 2×1 mg, triangles; 3×1 mg, circles), immediate release dosage form (inverted triangles, comparative control) or FAST dosage form (* symbols, 2×1 mg, comparative control);

FIGS. 10A-10C are graphs from an assessment of the sedation potential using the Tufts University benzodiazepine scale (FIG. 10A), Cole/ARCI sedation—mental (FIG. 10B), and Cole/ARCI sedation—motor (FIG. 10C) on subjects after Treatments A-F, corresponding respectively to placebo (Treatment A, diamonds), SLOW dosage form at dosages of 1 mg (Treatment B, squares), 2×1 mg (Treatment C, triangles), and 3×1 mg (Treatment D, circles), FAST controlled release (2×1 mg, Treatment E, * symbols), and immediate release (2 mg, Treatment F, inverted triangles);

FIGS. 11A-11B are plots of alprazolam plasma concentration, in ng/mL, as a function of time, in hours, on Day 1 (FIG. 11A) and on Day 6 (FIG. 11B) of a six day in vivo treatment with an immediate release alprazolam dosage form (1 mg tablet) taken orally every 8 hours for the six day test period (diamonds, Treatment 1) or with 3×1 mg SLOW alprazolam dosage forms taken orally once per day for the six day period (squares, Treatment 2); and

FIGS. 12A-12C show the results from Cognitive Drug Research's (CDR) computerized cognitive assessments after administration of alprazolam from an immediate release dosage form (Treatment 1, 1 mg taken orally every 8 hours for the six day test period, squares), from SLOW alprazolam dosage form (Treatment 2, 3×1 mg dosage form taken orally once per day for six days; triangles), and with a placebo SLOW alprazolam dosage form (Treatment 3; circles), for a Digit Symbol Substitution Test (DSST) (FIG. 12A), a Tracking Test (average distance from target, FIG. 12B), and a self-rated alertness (FIG. 12C) on days 1, 4, and 6 of the test.

### DETAILED DESCRIPTION OF THE INVENTION

I. Definitions

By “dosage form” is meant a pharmaceutical composition or device comprising an active pharmaceutical agent, such as alprazolam, the composition or device optionally containing inactive ingredients, i.e., pharmaceutically acceptable excipients such as suspending agents, surfactants, disintegrants, binders, diluents, lubricants, stabilizers, antioxidants, osmotic agents, colorants, plasticizers, coatings and the like, that are used to manufacture and deliver active pharmaceutical agents.

By “active agent”, “drug”, or “compound” is meant an agent, drug, or compound having the characteristics of alprazolam.

Reference to “alprazolam” includes the free base form of the drug and pharmaceutically-acceptable acid addition salt thereofs.

“Pharmaceutically-acceptable acid addition salts” or “pharmaceutically acceptable salts” are meant those salts in which the anion does not contribute significantly to the toxicity or pharmacological activity of the salt, and, as such, they are the pharmacological equivalents of the bases of the alprazolam compound. Examples of pharmaceutically acceptable acids that are useful for the purposes of salt formation include, but are not limited to, hydrochloric, hydrobromic, hydroiodic, citric, acetic, benzoic, mandelic, phosphoric, nitric, mucic, isethionic, palmitic, and others.

By “sustained release” is meant predetermined continuous release of active agent to an environment over a prolonged period.

The expressions “exit,” “exit orifice,” “delivery orifice” or “drug delivery orifice,” and other similar expressions, as may be used herein include a member selected from the group consisting of a passageway, an aperture, an orifice, and a bore. The expression also includes an orifice that is formed or formable from a substance or polymer that erodes, dissolves or is leached from the outer wall to thereby form an exit orifice.

A “dissolution rate” refers to the quantity of drug released in vitro from a dosage form per unit time into a release medium. In vitro dissolution rates in the studies described herein were performed on dosage forms placed in metal coil sample holders attached to a USP Type VII bath indexer in a constant temperature water bath at 37° C. Aliquots of the release rate solutions were injected into a chromatographic system to quantify the amounts of drug released during each testing interval.

The terms “in vitro release rate assay” or “in vitro dissolution assay” refer to a standardized assay for the determination of the quantity of drug released from a dosage form per unit time. For example, where the dosage form is an orally administrable controlled release dosage form, the release rate assay may be conducted using a USP Type 7 interval release apparatus. It is understood that reagents of equivalent grade may be substituted in such an assay in accordance with generally accepted procedures.

A drug “release rate” or “delivery rate” refers to the quantity of drug released in vivo from a dosage form or delivered per unit time, e.g., milligrams of drug released per hour (mg/hr) in vivo.

For clarity and convenience herein, the convention is utilized of designating the time of drug administration as zero hours (t=0 hours) and times following administration in appropriate time units, e.g., t=30 minutes or t=2 hours, etc.

As used herein, unless otherwise specified, a drug release rate obtained at a specified time “following administration” refers to the release rate obtained at the specified time following in vivo delivery of the dosage form. The time at which a specified percentage of the drug within a dosage form has been released may be referenced as the “Tₓ” value, where “x” is the percent of drug that has been released. For example, commonly used reference measurements for evaluating drug release from dosage forms are the time at which 70% of drug within the dosage form has been released and the time at which 90% of the drug within the dosage form has been released. These measurements are referred to as the “T₇₀” and the “T₉₀” for the dosage form.

An “immediate-release dosage form” refers to a dosage form that releases drug substantially completely within a short time period following administration, i.e., generally within a few minutes to about 1 hour.

By “sustained release dosage form” or “controlled release dosage form” is meant a dosage form that releases drug substantially continuously for several hours, typically for a period of at least about 10 to 20 hours and preferably 15 to 18 hours.

The term “uniform release rate” indicates an average hourly release rate that varies positively or negatively by no more than about 30%, preferably no more than about 25%, and most preferably no more than 10%, from either the preceding or the subsequent average hourly release rate as determined by any suitable release rate assay. For example, wherein the dosage form is an orally administrable controlled release tablet or capsule, the release rate performance of the dosage form can be evaluated using a USP Type 7 Interval Release Apparatus where the cumulative release is between about 25% to about 75%.

By “prolonged period of time” is meant a continuous period of time of at least about 4 hours, preferably 6-8 hours or more and, more preferably, 10 hours or more. For example, the exemplary osmotic dosage forms described herein generally begin releasing alprazolam at a uniform release rate within about 2 to about 6 hours following administration and the uniform rate of release, as defined above, continues for a prolonged period of time from about 25% to until at least about 75% and preferably at least about 85% of the drug is released from the dosage form. Release of alprazolam continues thereafter for several more hours although the rate of release is generally slowed somewhat from the uniform release rate.

By “C” is meant the concentration of drug in the plasma of a subject, generally expressed as mass per unit volume, typically nanograms per milliliter. For convenience, this concentration may be referred to as “plasma drug concentration” or “plasma concentration” herein which is intended to be inclusive of drug concentration measured in any appropriate body fluid or tissue. The plasma drug concentration at any time following drug administration is referenced as Cₜᵢₘₑ, as in C₉ₕ or C₂₄ₕ, etc. The term Cₘₐₓ refers to the maximum attained plasma drug concentration following administration of a drug dose, and is typically monitored after administration of a first dose and/or a non-continuous, non-steady state dosing regimen. “Tmax” refers to the time at which the maximum attained plasma drug concentration is achieved.

By “steady state” is meant a pattern of plasma concentration versus time following continuous administration of a constant dose, where the plasma concentration peaks and plasma concentration troughs are essentially identical within each dosing interval.

Persons of skill in the art appreciate that plasma drug concentrations obtained in individual subjects will vary due to interpatient variability in the many parameters affecting drug absorption, distribution, metabolism and excretion. For this reason, unless otherwise indicated, mean values obtained from groups of subjects are used herein for purposes of comparing plasma drug concentration data and for analyzing relationships between in vitro dosage form release rate assays and in vivo plasma drug concentrations.

II. Dosage Form Compositions and In vitro Release Profile

In a first aspect, the present invention provides a dosage form comprised of a desired dose of alprazolam, where the dosage form provides a specific alprazolam release profile as will be discussed and illustrated below. In general, the dosage form delivers alprazolam over an extended period of time such that once-a-day administration of the drug is possible. The dosage form also delivers alprazolam in a manner that results in relatively fewer and/or reduced side affects, as will be illustrated in the data presented below.

A. Dissolution Rates of Exemplary Dosage Forms

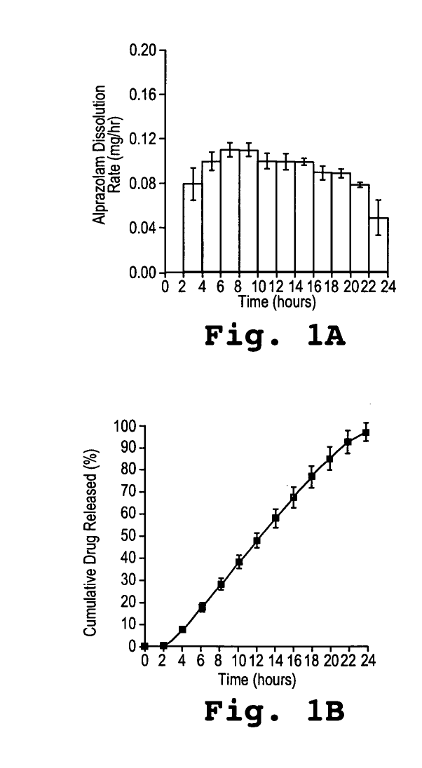

An exemplary dosage form containing two milligrams of alprazolam was prepared as described in Example 1. In brief, the dosage form was comprised of a drug layer and a push layer, surrounded by a semi-permeable membrane. Drug release is provided via an exit penetrating the semi-permeable membrane into the drug layer. In vitro release of alprazolam from the dosage forms was determined as described in Example 1 and is shown in FIGS. 1A-1B.

FIG. 1A shows the average amount of drug released per hour, in mg/hr, as a function of time. FIG. 1 B presents the data as normalized cumulative amount of drug released as a percent of total drug amount as a function of time, in hours. Four hours after exposure to the in vitro aqueous environment about 0.08 mg or about 4% of the dose was released. Ten hours after exposure to the in vitro aqueous environment about 40% of the total dose was released from the dosage form and at twelve hours about 50% of the total dose was released. Sixteen hours after exposure to the aqueous environment, less than 70% of the total dose was released, leaving 30% of the dose for delivery in the time frame between 16-24 hours. Ninety percent of the total dose was released 20.6 hours after exposure to the aqueous release medium and the average release rate was 0.102 mg/hr, calculated using the iterative method described in Example 1.

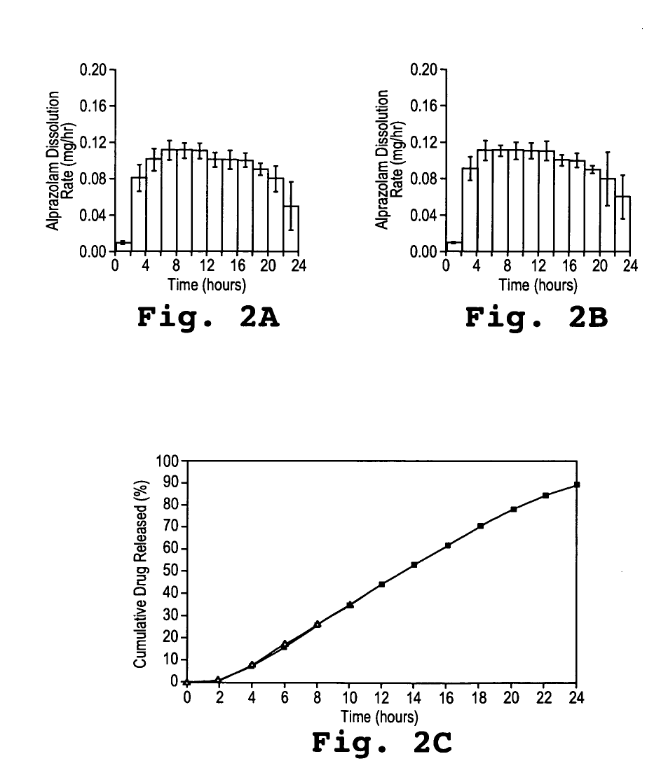

In another study, dosage forms comprising 2 mg of alprazolam were prepared as described in Example 2. Release of alprazolam from the dosage forms into medium simulating artificial gastric fluid (AGF, pH 1.2)and into artificial intestinal fluid (AIF, pH 6.8) was determined and the results are shown in FIGS. 2A-2C.

FIGS. 2A-2B show the average release rate in mg/hr of alprazolam from the dosage forms into artificial gastric fluid (FIG. 2A) and into artificial intestinal fluid (FIG. 2B). Release of alprazolam was unaffected by the pH of the release medium, with the dosage forms releasing 90% of the total dose (T₉₀) at 19.5 hours into artificial gastric fluid (FIG. 2A) and at 19.1 hours into artificial intestinal fluid (FIG. 2B). The average release rate into each fluid was 0.106 mg/hr and 0.104 mg/hr (calculated using the iterative method described in Example 1), respectively.

FIG. 2C shows the percent of the total alprazolam dose (2 mg) released over a 24 hour period. Presentation of the data in this format also shows that release of alprazolam was unaffected by the pH of the release medium, with the dosage forms having nearly identical release profiles whether placed in artificial gastric fluid (squares) or in artificial intestinal fluid (triangles). Two hours after exposure to the aqueous release medium, less than 10%, and more specifically only about 1% of the total drug dose was released. Four hours after exposure to the aqueous release medium, less than 10%, and more specifically about 8%, of the total drug dose was released. Approximately 35% of the drug dose was released after 10 hours of exposure to the aqueous medium and about 45% was released after 12 hours of exposure to the aqueous medium.

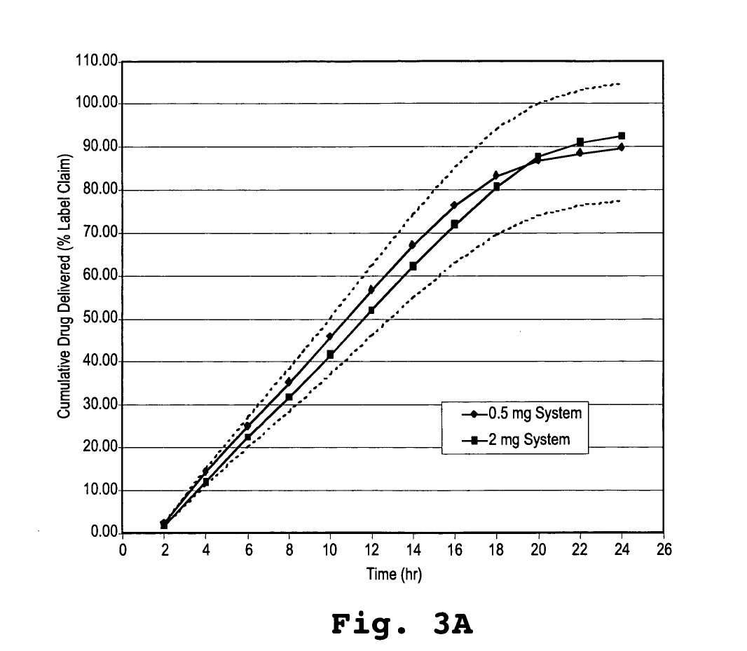

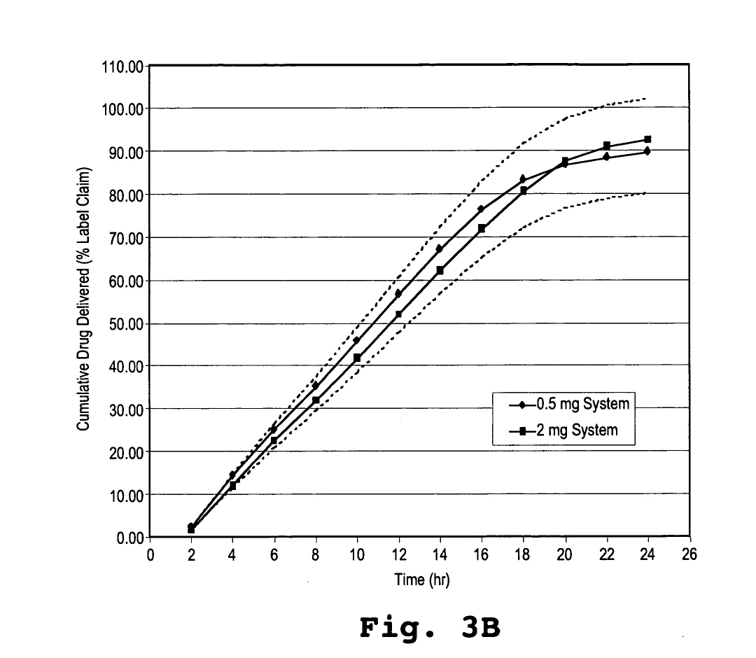

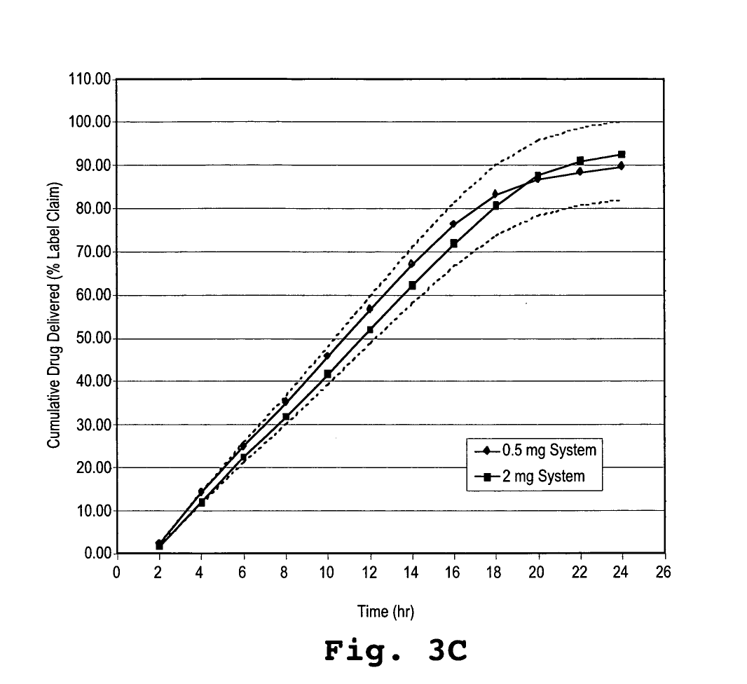

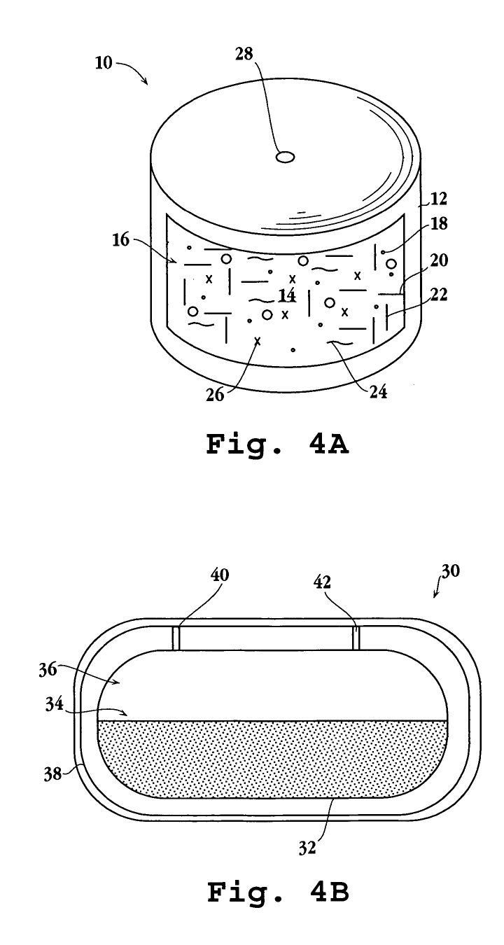

Additional dosage forms comprising 0.5 mg alprazolam and 2 mg alprazolam were prepared, as described in Example 3. Release of the drug was determined and the results are presented in FIGS. 3A-3C. FIG. 3A shows the in vitro amount of drug released (reported as percent total dose released at each time point) from the 0.5 mg (diamonds) and 2 mg (squares) dosage forms. The dashed lines in the figure correspond to the amount of alprazolam released at each time (averaged for the 0.5 mg and 2 mg dosage forms) plus and minus 15%. Two hours after exposure to the aqueous release medium about 2% of the total drug dose was released. Ten hours after exposure to the aqueous release medium about 42-46% of the total drug dose was released. Twelve hours after exposure to the aqueous release medium between about 52-57% of the total drug dose was released. More generally, a dosage form effective to provide an in vitro release profile where more than about 25% of the total dose and less than about 60% of the total drug dose, i.e., between 25-60%, is released by the ten hour time point reading in an in vitro release rate assay, averaged for at least about five dosage forms, the release assay conducted in accord with the protocol for a United States Pharmacopeia (USP) Type VII apparatus. In another general embodiment, a dosage form effective to provide an in vitro release profile where more than about 30% of the total dose and less than about 80% of the total drug dose, i.e., between 30-80%, is released ten hours after initiation of an in vitro release rate assay, is contemplated.

In another general embodiment, a dosage form effective to provide a dissolution profile where more than about 35% of the total dose and less than about 55% of the total drug dose, i.e., between 35-55%, is released at the ten hour time point reading in an in vitro release rate assay is contemplated. This embodiment is shown in FIG. 3B, where the release data for the 0.5 and 2 mg dosage forms is shown with dashed lines representing plus and minus 12% of the averaged amount of drug released at each time point for the two dosage forms. Dosage forms capable of providing a release profile bounded by the dashed lines in FIG. 3B are contemplated herein. More specifically, a dosage form that releases more than 35% and less than 55% of the total drug dose ten hours after contact with an aqueous medium is provided. More preferably, a dosage form that releases more than 40% and less than 50% of the total drug dose ten hours after contact with an aqueous medium is provided.

FIG. 3C shows another embodiment where the cumulative amount of alprazolam released as a function of time for the 0.5 and 2 mg dosage forms is shown, with dashed lines representing plus and minus 10% of the averaged amount of drug released at each time point. Dosage forms capable of providing a dissolution rate bounded by the dashed lines in FIG. 3C are contemplated herein. More specifically, a dosage form that releases at least about 40% and less than 50% of the total drug dose ten hours after contact with an aqueous medium is provided. More preferably, a dosage form that releases at least about 40% and less than 46% of the total drug dose ten hours after contact with an aqueous medium is provided.

The data in FIGS. 1-3 illustrate that the dosage form is effective to provide the desired release profile essentially independently of the external milieu. The dissolution profile of the dosage form was substantially constant regardless of the release medium, as is apparent from the data in FIGS. 1-3 where the pH of the external release medium was varied. Thus, the dosage form provides an in vivo release profile essentially the same as the dissolution release profile. Accordingly, in another aspect, a dosage form that provides a release profile where between 25-60% of the total dose is released 10 hours after exposure to an aqueous environment, e.g., after oral ingestion of the dosage form, is contemplated. In one embodiment, the dosage form releases alprazolam at a rate sufficient to achieve between 35-55% of the dose 10 hours after delivery. Alternatively, the dosage form releases between 30-80% of the dose 12 hours after oral delivery.

B. Exemplary Dosage Forms

The dosage form of the present invention may be configured and formulated according to any design that delivers a desired dose of alprazolam according to the release profiles exemplified in FIGS. 1-3. Typically, the dosage form is orally administrable and is sized and shaped as a conventional tablet or capsule. Orally administrable dosage forms may be manufactured according to one of various different approaches. For example, the dosage form may be manufactured as a diffusion system, such as a reservoir device or matrix device, a dissolution system, such as encapsulated dissolution systems (including, for example, “tiny time pills”, and beads) and matrix dissolution systems, or combination diffusion/dissolution systems and ion-exchange resin systems, as described in Remington's Pharmaceutical Sciences, 1990 ed., pp. 1682-1685. In a preferred embodiment, the dosage form is an orally administrable, osmotically driven dosage form, as will now be described.

Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. An advantage to osmotic systems is that their operation is pH-independent, as illustrated above with respect to FIGS. 2A-2C, and thus continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release, 35:1-21 (1995). Osmotic dosage forms are also described in detail in the following U.S. Patents, each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

In brief, an osmotic dosage form 10 can be of the configuration shown in FIG. 4A. Dosage form 10, shown in a cutaway view, is also referred to as an elementary osmotic pump, and is comprised of a semi-permeable wall 12 that surrounds and encloses an internal compartment 14. The internal compartment contains a single component layer referred to herein as a drug layer 16, comprising alprazolam 18 in an admixture with selected excipients. The excipients are adapted to provide an osmotic activity gradient for attracting fluid from an external environment through wall 12 and for forming a deliverable alprazolam formulation upon imbibition of fluid. The excipients may include a suitable suspending agent, also referred to herein as drug carrier 20, a binder 22, a lubricant 24, and an osmotically active agent referred to as an osmagent 26. Exemplary materials for each of these components are provided below.

Semi-permeable wall 12 of the osmotic dosage form is permeable to the passage of an external fluid, such as water and biological fluids, but is substantially impermeable to the passage of components in the internal compartment. Materials useful for forming the wall are essentially nonerodible and are substantially insoluble in biological fluids during the life of the dosage form. Representative polymers for forming the semi-permeable wall include homopolymers and copolymers, such as, cellulose esters, cellulose ethers, and cellulose ester-ethers. Flux-regulating agents can be admixed with the wall-forming material to modulate the fluid permeability of the wall. For example, agents that produce a marked increase in permeability to fluid such as water are often essentially hydrophilic, while those that produce a marked permeability decrease to water are essentially hydrophobic. Exemplary flux regulating agents include polyhydric alcohols, polyalkylene glycols, polyalkylenediols, polyesters of alkylene glycols, and the like.

In operation, the osmotic gradient across wall 12 due to the presence of osmotically-active agents causes gastric fluid to be imbibed through the wall, swelling of the drug layer, and formation of a deliverable alprazolam formulation (e.g., a solution, suspension, slurry or other flowable composition) within the internal compartment. The deliverable alprazolam formulation is released through an exit 28 as fluid continues to enter the internal compartment. Even as drug formulation is released from the dosage form, fluid continues to be drawn into the internal compartment, thereby driving continued release. In this manner, alprazolam is released in a sustained and continuous manner over an extended time period.

FIG. 4B shows a schematic illustration of another exemplary osmotic dosage form. Dosage form 30, shown in cross-section, has a semi-permeable wall 32 defining an internal compartment 34. Internal compartment 34 contains a bilayered-compressed core having a drug layer 36 and a push layer 38. As will be described below, push layer 38 is a displacement composition that is positioned within the dosage form such that as the push layer expands during use, the materials forming the drug layer are expelled from the dosage form via one or more exit ports, such as exits 40,42. The push layer can be positioned in contacting layered arrangement with the drug layer, as illustrated in FIG. 4B, or can have one or more intervening layers separating the push layer and drug layer.

Drug layer 36 comprises alprazolam in an admixture with selected excipients, such as those discussed above with reference to FIG. 4A. In the dosage forms prepared for the studies discussed with respect to FIGS. 1-3, the drug layer was comprised of alprazolam, a poly(ethylene oxide) as a carrier, sodium chloride as an osmagent, hydroxypropylmethylcellulose as a binder, and magnesium stearate as a lubricant (see Examples 1-2). In one embodiment, a dosage form having a drug layer that excludes formulations consisting of two viscosity grades of hydroxypropylmethylcellulose is contemplated. In another embodiment, a dosage form having a drug layer that includes hydroxypropylmethylcellulose having a single viscosity or molecular weight is contemplated.

Push layer 38 comprises osmotically active component(s), such as one or more polymers that imbibes an aqueous or biological fluid and swells, referred to in the art as an osmopolymer. Osmopolymers are swellable, hydrophilic polymers that interact with water and aqueous biological fluids and swell or expand to a high degree, typically exhibiting a 2-50 fold volume increase. The osmopolymer can be non-crosslinked or crosslinked, and in a preferred embodiment the osmopolymer is at least lightly crosslinked to create a polymer network that is too large and entangled to easily exit the dosage form during use. Examples of polymers that may be used as osmopolymers are provided in the references noted above that describe osmotic dosage forms in detail. A typical osmopolymer is a poly(alkylene oxide), such as poly(ethylene oxide), and a poly(alkali carboxymethylcellulose), where the alkali is sodium, potassium, or lithium. Additional excipients such as a binder, a lubricant, an antioxidant, and a colorant may also be included in the push layer. In use, as fluid is imbibed across the semi-permeable wall, the osmopolymer(s) swell and push against the drug layer to cause release of the drug from the dosage form via the exit port(s).

The push layer can also include a component referred to as a binder, which is typically a cellulose or vinyl polymer, such as poly-n-vinylamide, poly-n-vinylacetamide, poly(vinyl pyrrolidone), poly-n-vinylcaprolactone, poly-n-vinyl-5-methyl-2-pyrrolidone, and the like. The push layer can also include a lubricant, such as sodium stearate or magnesium stearate, and an antioxidant to inhibit the oxidation of ingredients. Representative antioxidants include, but are not limited to, ascorbic acid, ascorbyl palmitate, butylated hydroxyanisole, a mixture of 2 and 3 tertiary-butyl-4-hydroxyanisole, and butylated hydroxytoluene.

An osmagent may also be incorporated into the drug layer and/or the push layer of the osmotic dosage form. Presence of the osmagent establishes an osmotic activity gradient across the semi-permeable wall. Exemplary osmagents include salts, such as sodium chloride, potassium chloride, lithium chloride, etc. and sugars, such as raffinose, sucrose, glucose, lactose, and carbohydrates.

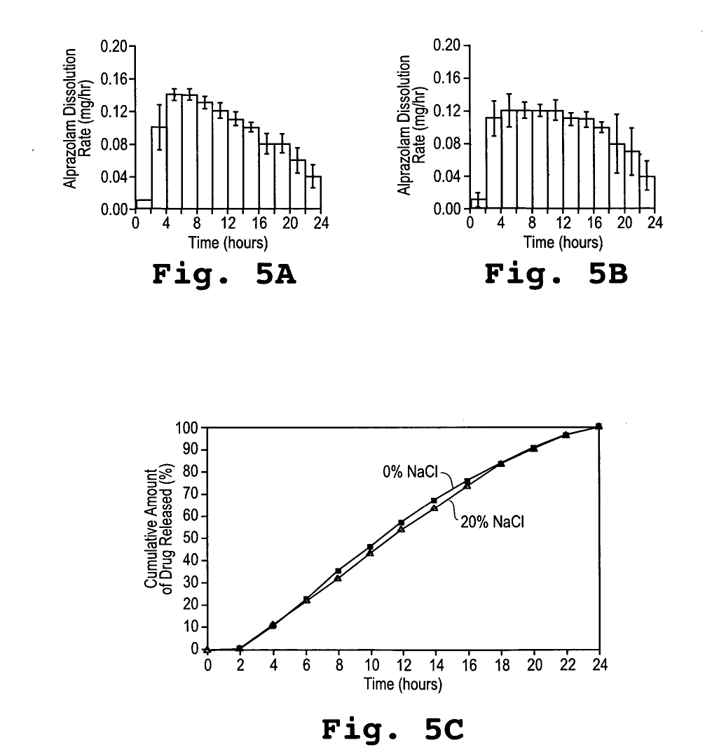

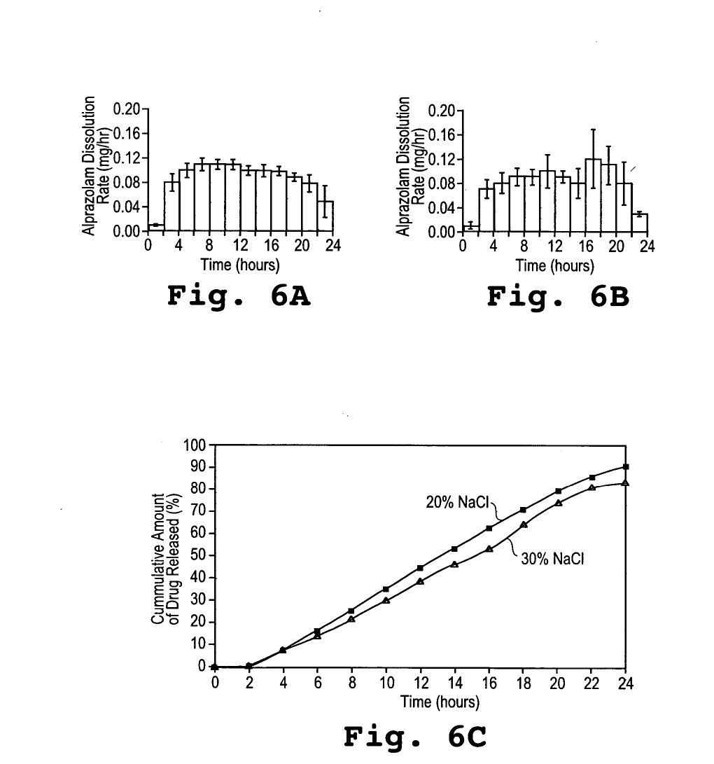

A study was conducted in support of the invention where the osmagent content in the drug layer was varied from 0% to 20% to 30%. Dosage forms having four compositions were prepared as described in Example 4. Two of the dosage forms contained 20% sodium chloride, differing only in the thickness of the semi-permeable membrane (see Examples 4A, 4B). Release of alprazolam from the dosage forms was determined in vitro and the results are shown in FIGS. 5A-5C and 6A-6C.

FIGS. 5A-5B show the in vitro release rate in mg/hr of alprazolam for the dosage forms prepared as described in Example 4A; specifically, dosage forms without sodium chloride in the drug layer (FIG. 5A) and with 20% sodium chloride in the drug layer (FIG. 5B). The release data is presented in FIG. 5C as cumulative amount of drug release normalized by the total drug dose (i.e., percent drug released) for the dosage form without sodium chloride in the drug layer (squares) and with 20% sodium chloride in the drug layer (triangles). Both formulations provided a release profile where ten hours after exposure to an aqueous environment, less than 60% of the total dose and more than about 25% of the total dose was released. More specifically, in the time interval beginning when the dosage forms were placed in contact with an aqueous environment to 10 hours after such contact, i.e., at the 10 hour time point, the dosage forms released about 45-48% of the drug load. At the twelve hour time point, 55-58% of the total drug dose was released into the aqueous medium. A comparison of FIG. 5A to FIG. 5B suggests that the presence of an osmagent in the drug layer results in a dosage form with a more uniform rate of release, as evident by the small fluctuation in amount of drug released per hour in the interval between 4-16 hours.

FIGS. 6A-6C show the release profiles of osmotic dosage forms prepared as described in Example 4B, where the drug layer was formulated to include 20% sodium chloride (FIG. 6A; FIG. 6C, squares) or 30% sodium chloride (FIG. 6B; FIG. 6C, triangles). The dosage form with 20% sodium chloride provided a uniform release rate, particularly between the interval of 4-20 hours after. exposure to an aqueous medium. Both dosage forms were effective to provide a release profile where, as seen in FIG. 6C, after exposure to an aqueous environment for about 10 hours, less than 60% of the total dose and more than about 25% of the total dose was released. More specifically, and with specific reference to the data for the dosage forms of FIGS. 6A-6C, at the 10 hour time point the dosage forms released about 30-35% of the drug load. At the twelve hour time point, 39-42% of the total drug dose was released into the aqueous medium. The slow, sustained release of alprazolam from the dosage forms offers considerable clinical benefits, as will be discussed below.

Referring back to FIG. 4B, the dosage form can optionally include an overcoat 44 for color coding the dosage forms according to dose. While the dosage form may include an overcoat for color coding, the optional overcoat, in one embodiment, does not contain alprazolam. Thus, in one embodiment, an orally administrable dosage form configured to deliver a therapeutically effective dose of alprazolam according to the release profiles illustrated above without the need for an optional drug overcoat is provided. In this embodiment, the dosage form excludes an overcoat that contains alprazolam. Without a drug-containing overcoat, the dosage form does not provide for an immediate release of drug upon administration by virtue of an amount of drug contained in on the external surface of the dosage form.

The preparation of osmotic dosage forms is well described in the art (see, for example U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850) and is illustrated in Examples 1-3 provided herein.

From the foregoing in vitro release studies, it is apparent that the invention provides a dosage form with a release profile that permits once daily dosing of alprazolam. The release profiles shown in FIGS. 1-3 and 5-6 provide a dosage form where (i) less than 20% of the dose is released 2 hours after exposure to an aqueous environment; (ii) between 25% and 60% of the dose is released after exposure to an aqueous environment for a time of about 10 hours; and (iii) greater than 85% of the dose is released after exposure to an aqueous environment for a time of about 24 hours. The dosage form is intended to provide a therapeutically effective plasma concentration for a prolonged period, and thus provides that at least about 10%, more preferably 15%, and still more preferably 20% of the total alprazolam dose is released into the external environment of use at times longer than 16 hours after delivery, e.g., after exposure of the dosage form to an aqueous environment. Alternatively, the dosage form provides for release of at least about 15%, more preferably 20%, of the total alprazolam dose 14 hours after exposure of the dosage form to an aqueous environment. Alternatively, the dosage form provides for release of at least about 25%, more preferably 30%, of the total alprazolam dose 12 hours after exposure of the dosage form to an aqueous environment.

In another embodiment, the dosage form is configured to release alprazolam at a rate that ranges between 2% of the total dose of alprazolam per hour to 7% of the total dose of alprazolam per hour over the period of between 2 and 20 hours, preferably 2 and 16 hours, still more preferably 2 and 12 hours, after exposure to an aqueous environment.

III. In vivo Characterization of Alprazolam Dosage Forms

An in vivo study was conducted to evaluate the pharmacodynamics of alprazolam delivered from the dosage forms described herein relative to an immediate release dosage form and to another controlled release dosage form having a different release profile from that provided by the present dosage form. Two osmotic dosage forms were prepared, referred to herein as a SLOW dosage form and as a FAST dosage form. The specific composition of the SLOW and the FAST dosage forms is provided in Example 5A, and in brief, the dosage forms both contained 1 mg of alprazolam and differed only in the thickness of the semipermeable wall. In in vitro release assays, the SLOW dosage form had a T₉₀ of 20 hours and the FAST dosage form had a T₉₀ of 10 hours.

As described in Example 5B, adults with a history of sedative or tranquilizer abuse were enrolled for a double-blinded, single-dose, study. Each subject enrolled (n=24) received five of six treatments, identified as Treatments A-F:

| | |
|---|---|
| Treatment A | placebo (SLOW¹ dosage form with no |
| | alprazolam) |
| Treatment B | SLOW¹ alprazolam; 1 mg |
| Treatment C | SLOW¹ alprazolam; 2 × 1 mg |
| Treatment D | SLOW¹ alprazolam; 3 × 1 mg |
| Treatment E | comparative control: FAST² alprazolam; |
| | 2 × 1 mg |
| Treatment F | comparative control: immediate- |
| | release alprazolam³; 2 mg |

¹SLOW dosage form composition provided in Example 5A.
²FAST dosage form composition provided in Example 5A.
³immediate-release alprazolam available under the tradename XANAX ®.

The treatments were separated by a washout period of not less than 4 days and not more than 21 days.

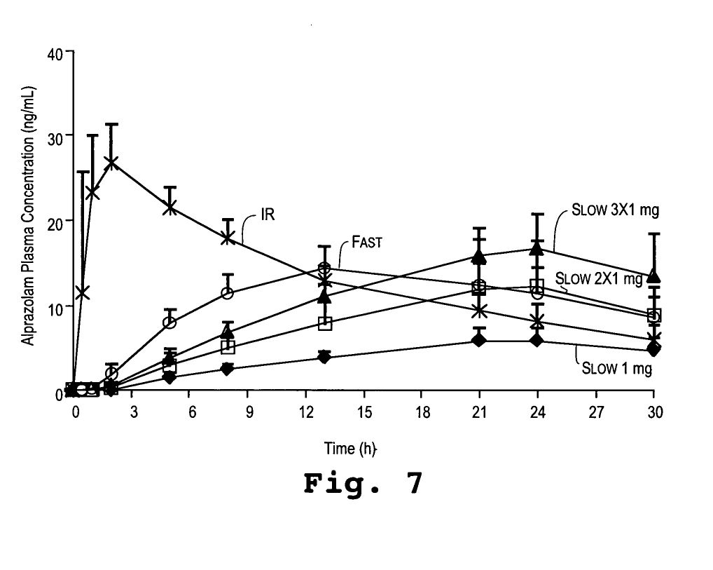

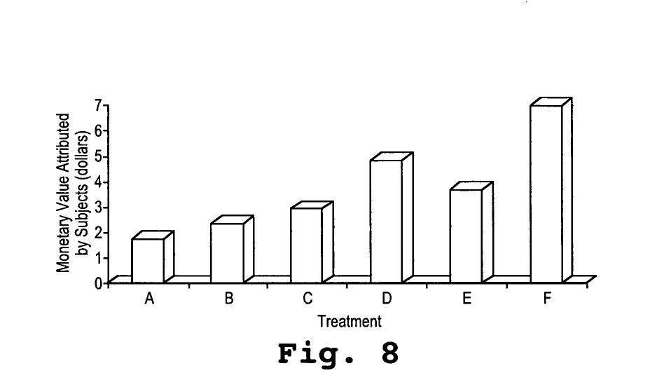

Plasma samples were collected predose and at defined intervals for 30 hours after dosing. FIG. 7 shows the alprazolam concentration, in ng/mL, over the 30 hour study period for the SLOW dosage form at dosages of 1 mg (diamonds), 2×1 mg (squares), and 3×1 mg (triangles), and for the comparative controls (i) FAST controlled release (x symbols) and (ii) immediate release (* symbols). There was a dose proportional increase in the alprazolam plasma concentration over the 1 to 3 mg dose range. The peak concentration resulting from the 3 mg SLOW dosage form (triangles) was lower than the peak plasma concentration resulting from the 2 mg immediate release conventional dosage form (* symbols). Referring to Table 1, peak concentrations were noted at approximately 21 to 23 hours for the SLOW system, 15 hours for the FAST system, and within about 2 hours for the immediate release formulation. The pharmacokinetic results for Treatments B-F are summarized in Table 1.

TABLE 1
Pharmacokinetic Parameters Following
Single-Dose Alprazalom Treatments

| | | | | | TREATMENT F |
|---|---|---|---|---|---|
| | Treatment B | Treatment C | Treatment D | Treatment E | (immediate |
| Parameter | (SLOW 1 × 1 mg) | (SLOW 2 × 1 mg) | (SLOW 3 × 1 mg) | (FAST 2 × 1 mg) | release, 2 mg) |
| Cₘₐₓ (ng/mL) | 6.90 ± 1.6 | 13.3 ± 5.1 | 18.5 ± 4.1 | 15.9 ± 3.0 | 28.9 ± 5.6 |
| Tₘₐₓ (hr) | 22.8 ± 3.9 | 21.4 ± 5.1 | 23.3 ± 1.5 | 14.9 ± 4.7 | 2.29 ± 1.7 |
| Cₘₐₓ/D | 6.9 | 6.7 | 6.2 | 8.0 | 14.5 |
| Cₘₐₓ/Tₘₐₓ | 0.303 | 0.621 | 0.794 | 1.07 | 12.6 |
| Cₘₐₓ/Tₘₐₓ | 0.303 | 0.311 | 0.298 | 0.535 | 6.3 |
| D | | | | | |
| AUC | 88.6 | 167.8 | 240.9 | — | — |
| (ng*hr/mL) | | | | | |
| AUC | 88.6 | 83.9 | 80.3 | — | — |
| D | | | | | |
| (ng*hr/mL*mg) | | | | | |

Based on the data in Table 1 and FIG. 7, the SLOW dosage form described herein provides a maximum attained plasma concentration (Cₘₐₓ) more than 13 hours after administration of the dosage form to a subject, more preferably more than 14 hours, and still more preferably greater than 16 hours, and even more preferably greater than 20 hours after administration. The dosage form described herein also provides a dose normalized ratio of maximum attained alprazolam plasma concentration (Cₘₐₓ) to time to reach maximum attained alprazolam plasma concentration (Tₘₐₓ) of less than 0.5.

The dosage form described herein can also be characterized by a release rate that results in a Cₘₐₓ that is less than twice the plasma concentration at 24 hours (C₂₄) and occurs more than about 13 hours or 16 hours after administration. In preferred embodiments, the dosage form of the present invention is characterized by a Cₘₐₓ that is less than twice the C₂₄ and occurs more than 18 hours after administration. In particularly preferred embodiments, the dosage form of the present invention is characterized by a Cₘₐₓ that is less than twice the C₂₄ and occurs more than 20 hours, preferably more than 22 hours, after administration.

As FIG. 7 shows, the plasma concentrations provided by the dosage form will vary with the dose of alprazolam included in the dosage form. Dosage forms providing an alprazolam dose of between 0.25-25 mg, more preferably 0.5-20 mg, are contemplated. Such dosage forms are characterized by dose normalized plasma concentrations ranging from 1 ng/mL*mg to 8 ng/mL*mg, with dose normalized plasma concentrations ranging from 4 ng/mL*mg to 6 ng/mL*mg being more typical. As the dose of alprazolam increases, the dosage form is preferably characterized by a Cₘₐₓ that increases by less than 10 ng/mL per mg increase in the alprazolam dose contained in the dosage form, with an increase in Cₘₐₓ of less than 8 ng/mL per mg being even more preferred.

The dose-adjusted area under the curve (AUC) for the dosage forms described herein ranged from about 80-87 ng·hr/mL·mg. In one embodiment, the invention provides a dosage form where the AUC is less than 110 ng·hr/mL·mg, more preferably less than 100 ng·hr/mL·mg. Typically, the AUC is between about 70-110 ng·hr/mL·mg, more preferably between 75-100 ng·hr/mL·mg.

The dosage form can be further or alternatively characterized by controlled release of alprazolam at a rate that provides a quotient of (Cₘₐₓ/Tₘₐₓ)/D, wherein “D” equals the dose of alprazolam. Preferably, the quotient of (Cₘₐₓ/Tₘₐₓ)/D provided by such an embodiment is 1.0 or less, with dosage forms that provide a quotient of (Cₘₐₓ/Tₘₐₓ)/D that is 0.5 or less being particularly preferred.

With continuing reference to the in vivo study described in Example 5, the pharmacodynamic effects of the:treatment regimens (Treatments A-F, see table above and in Example 5) were evaluated using various assessments of the potential for abuse liability. In one assessment, the test subjects were asked to attribute a monetary value to receive an additional dose of the drug. The monetary value provides a ranking of the likelihood of the drug formulation being abused. FIG. 8 shows the results of this-assessment, where the dollar value attributed by the test subjects in each treatment group is shown. As can be seen, the subjects ascribed the highest monetary value to the immediate release, control-formulation (Treatment F). The difference between this formulation and the placebo (Treatment A) was statistically significant. However, there was no statistical difference in the monetary value between the placebo (Treatment A) and the SLOW dosage forms described herein (Treatments B, C, D; dosages of 1 mg, 2×1 mg and 3×1 mg, respectively).

A drug effects questionnaire was completed by the test subjects to assess their subjective views on the strength of the dosage formulation in each treatment group, their liking of the dosage form, and whether they would take it again. The results are tabulated in Table 2, where data collected in Treatments B-F are reported as a difference of Treatment A.

TABLE 2
Summary of Drug Effects Questionnaire
(Data Reported are Estimate of Difference (p-value))

| | Trmt B − Trmt A | Trmt C − Trmt A | Trmt D − Trmt A | Trmt E − Trmt A | Trmt F − Trmt A |
|---|---|---|---|---|---|
| Strength | −0.49 | −0.75 | 13.15 | 11.67 | 27.92 |
| | (0.913) | (0.864) | (0.003) | (0.007) | (<0.001) |
| Liking | −2.69 | −4.16 | 8.04 | −1.66 | 10.16 |
| | (0.425) | (0.208) | (0.017) | (0.610) | (0.002) |
| Take | −0.23 | −10.45 | 2.54 | −1.97 | 12.64 |
| Again | (0.557) | (0.003) | (0.478) | (0.570) | (<0.001) |

Treatment A = Placebo; Treatment B = 1 mg SLOW; Treatment C = 2 × 1 mg SLOW; Treatment D = 3 × 1 mg SLOW; Treatment E = 2 × 1 mg FAST; Treatment F = 2 mg immediate release

The negative values in Table 2 for treatment differences compared with placebo (Treatment A) indicate that treatment with an alzprazolam-containing dosage form (Treatment B-Treatment A) did not have a greater effect than placebo. In all instances, subjects rated the immediate release dosage form (Treatment F) higher in strength than placebo and that they liked the effect provided by the immediate release dosage form and would take it again. In contrast, there was no difference in the strength and liking between the 1 mg and 2 mg SLOW dosage forms (Treatments B, C respectively) and placebo. In fact, subjects indicated that they would avoid the 2 mg SLOW (Treatment C) system. A comparison of the treatments using a dosage form that provided a two milligram dose, Treatments C (SLOW dosage form), Treatment E (FAST dosage form), and Treatment F (immediate release dosage form), shows that patients found the immediate release dosage form highest in strength relative to the placebo, liked it the best and would take it again. A two milligram dose delivered from the FAST dosage form was rated by the patients as less strong than the immediate release with no particular desire to take it again, relative to the placebo. A two milligram dose provided from the SLOW dosage form was rated as not being different in strength than the placebo, with no particular liking or desire for taking it again. The data also indicates that the 1 milligram dose from the SLOW dosage form (Treatment B) and the 2 milligram dose from the SLOW dosage form (Treatment C) both provided a system that is less likely to result in abuse by the patient, as users rated these dosage forms as being of less strength, as liking them less, and as being less likely to take them again.

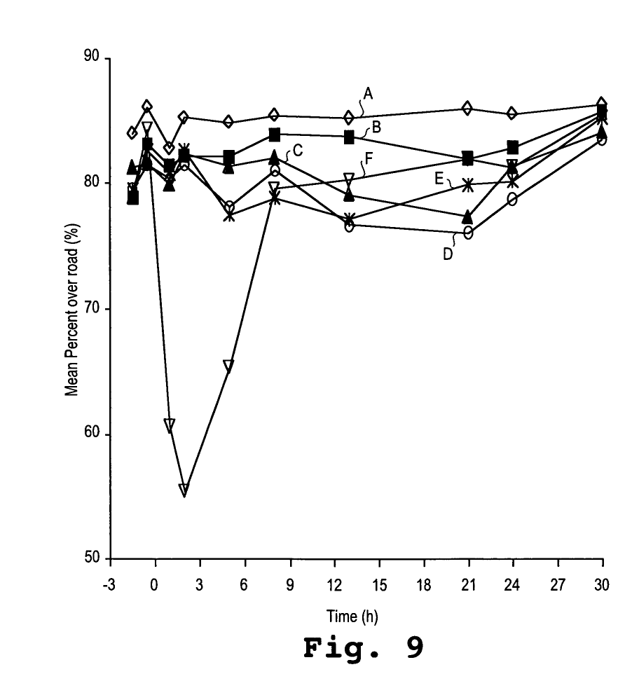

The potential for psychomotor impairment resulting from each dosage form was assessed using a standardized manual tracking test where subjects, on a computer simulator, attempted to keep a constant speed and steady lateral position of a vehicle between delineated lines. During the test, the computer captured how much time the subject spent away from the lateral position, i.e, time off-the-road, and how far off the road the vehicle went. The performance results are reported as a percent over-the-road value and are shown in FIG. 9. In subjects treated with a placebo (Treatment A, diamonds), a mean percent over-the-road of around 85% was observed. In the subjects dosed with alprazolam from the SLOW dosage forms (Treatment B, 1 mg, squares; Treatment C, 2×1 mg, triangles; Treatment D, 3×1 mg, circles), it was observed that the subjects were able to maintain the vehicle in the desired lateral position a higher percentage of the time than those treated with the immediate release alprazolam dosage form (Treatment F, 2 mg, inverted triangles). The highest dose of alprazolam (3×1 mg, Treatment D) delivered to the subjects from the SLOW dosage form gave results similar to that observed in the patients treated with the lower dose (2 mg) FAST dosage form (Treatment E, * symbols). The values from this tracking test are presented in Table 3A in Example 5B.

Table 3B in Example 5B shows the results of a second test for psychomotor assessment, a digital symbol substitution test, where after dosing subjects are given a test involving the substitution of simple figures/symbols for digits. In the test, a series of randomized digits are presented and the subject draws a symbol below each digit as indicated by a code presented with each digit. The number of correct symbols substituted for digits during a two minute period is measured. The data in Tables 3A-3B show that alprazolam (2 mg dose) administered from an immediate release dosage form resulted in a consistent impairment in psychomotor function. In contrast, few observations of psychomotor impairment were observed when alprazolam was administered from the SLOW dosage form, with only a minor impairment with the 2 mg dose in the tracking test.

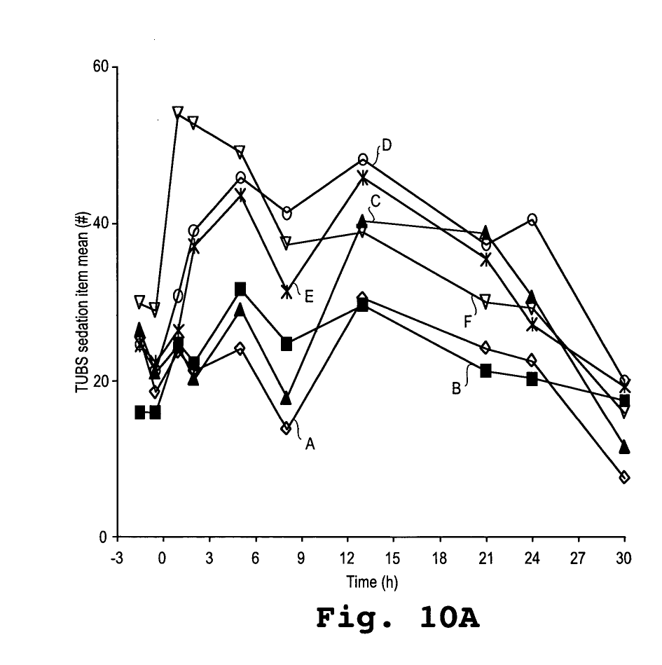

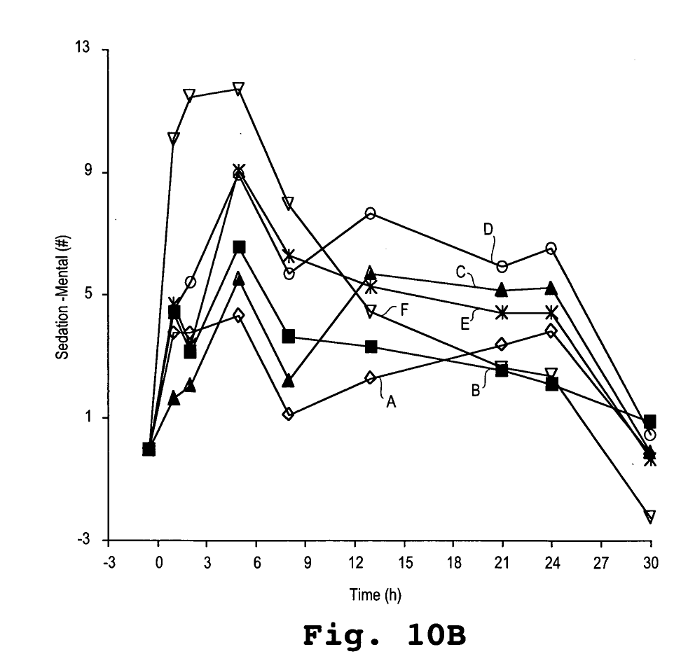

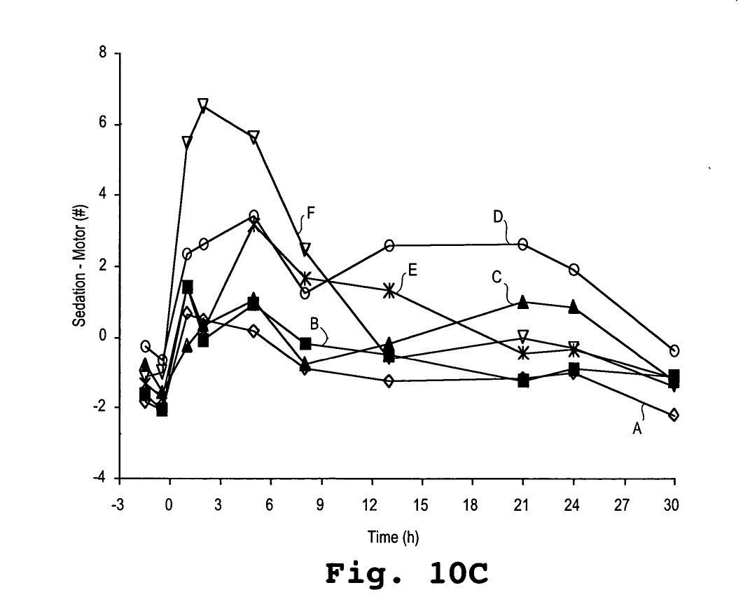

To evaluate the potential for sedation, an additional complement of tests was conducted, as described in Example 5B. The results from three of the tests are presented in FIGS. 10A-10C, and Table 4 below summarizes the data from all six tests performed. FIG. 10A shows the results from an assessment of the sedation potential using the Tufts University benzodiazepine scale. FIG. 10B shows the results from the Cole/ARCI sedation—mental assessment and FIG. 10C corresponds to the results obtained using the Cole/ARCI sedation—motor test. The subjects were evaluated as a function of time after dosing according to one of Treatments A-F, corresponding respectively to placebo (Treatment A, diamonds), SLOW dosage form at dosages of 1 mg (Treatment B, squares), 2×1 mg (Treatment C, triangles), and 3×1 mg (Treatment D, x symbols), FAST controlled release (2×1 mg, Treatment E, * symbols), and immediate release (2 mg, Treatment F, circles). The data shows that subjects were less sedated with the SLOW formulation, particularly at dosages of 1-2 mg in the interval of 1-8 hours after delivery, than with the immediate release dosage form and the FAST dosage form.

Accordingly, the invention provides a method for reducing the side effects associated with oral delivery of alprazolam by administering a dose of alprazolam in a dosage form that provides a Cₘₐₓ more than 14 hours after administration, more preferably more than 16 hours after administration. FIGS. 10A-10C illustrate that such a dosage form provides a reduction in sedation, preferably a reduction of at least about 2-fold, more preferably at least 3-fold, as measured by a TUBS assessment or a Cole/ARCI sedation-mental assessment 2 hours after administration, relative to the same dose of alprazolam administered in an immediate release dosage form.

Table 5 summarizes the adverse events reported in the in vivo study. The adverse events of somnolence, dizziness, or an abnormal gait following treatment with alprazolam from the Treatments A-F were recorded.

TABLE 5
Percent of Adverse Events Following
Single-Dose Alprazalom Treatments

| | Treatment | Treatment | Treatment | Treatment | Treatment | Treatment |
|---|---|---|---|---|---|---|
| | A | B | C | D | E | F |
| Adverse | (placebo) | (SLOW 1 × 1 mg) | (SLOW 2 × 1 mg) | (SLOW 3 × 1 mg) | (FAST 2 × 1 mg) | (immediate release, 2 mg) |
| Event | N = 18 | N = 17 | N = 17 | N = 17 | N = 18 | N = 16 |
| Somnolence | 11.1% (2)¹ | 5.9% (1) | 11.8% (2) | 5.9% (1) | 22.2% (4) | 31.3% (5) |
| Dizziness | 0 | 0 | 0 | 0 | 5.6% (1) | 25% (4) |
| Abnormal gait | 0 | 0 | 0 | 0 | 0 | 25% (4) |

¹value in parenthesis is actual number of subjects reporting the adverse event.

The highest incidence of side effects was noted with the immediate release dosage form (Treatment F). Nervous system-related side effects included somnolence, dizziness, and abnormal gait. These results indicate that the incidence of side effects appears to increase as the rate of release of alprazolam increases. The side effects were also significant on the first day of therapy with the immediate release formulation. Although such side effects may abate as treatment with an immediate release dosage form continues, the high incidence of side effects early in a treatment program often impairs a patient's ability to function normally. The side effects were much lower with the SLOW controlled release formulations (Treatments B, C, D) and the reduction in side effects allows patients to more easily continue normal activities.

A second in vivo study was conducted, where subjects were treated with a multi-dosing regimen of alprazolam dosage forms. As described in Example 6, 36 healthy subjects received three treatment regimens sequentially, identified as Treatment 1, 2, and 3, with a washout period between regimens. All treatment regimens were for six days. Treatment 1 corresponded to an immediate release alprazolam, 1 mg tablet delivered orally every 8 hours for the six day test period. Treatment 2 corresponded to 3×1 mg SLOW alprazolam administered orally once per day for the six day period. Treatment 3 involved oral administration of three placebo SLOW dosage forms once per day for the six days. During the treatment period, blood samples were drawn for analysis and various cognative assessments were given.

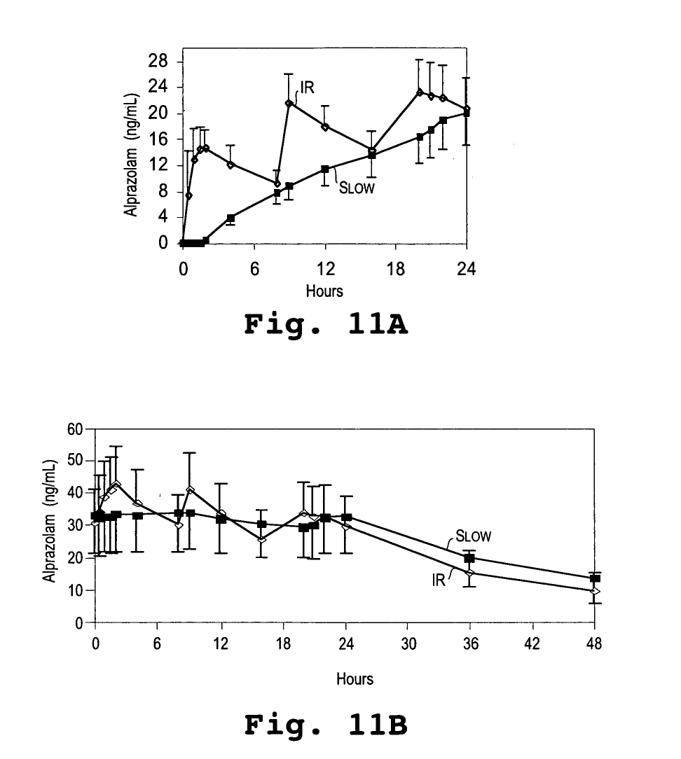

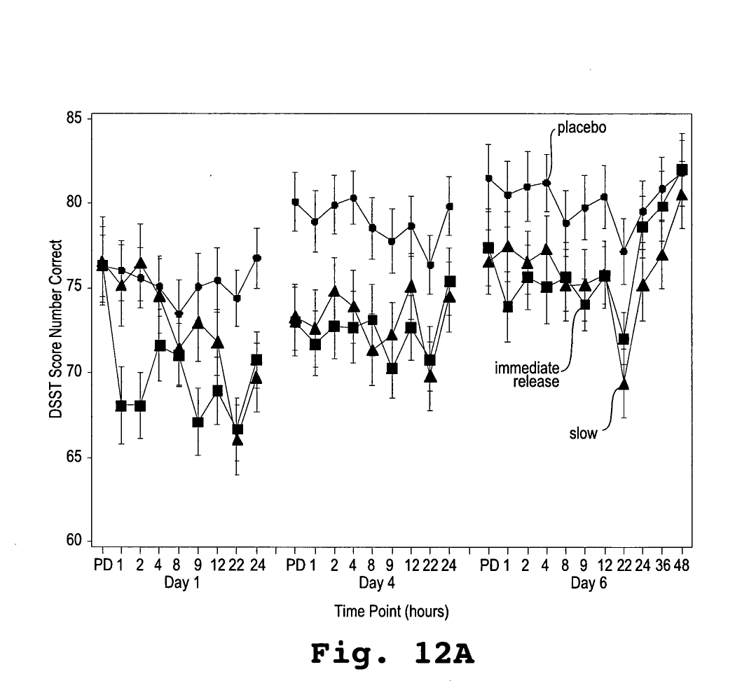

FIGS. 11A-11B show the alprazolam plasma concentrations on Day 1 (FIG. 11A) and Day 6 (FIG. 11B) for the subjects receiving Treatment 1 (immediate release alprazolam dosage form, diamonds) and Treatment 2 (SLOW alprazolam dosage form, squares). On Day 1, the drug plasma concentration resulting from the SLOW alprazolam dosage form (squares) was significantly more uniform than that provided by the immediate release dosage form (diamonds). The Cₘₐₓ for the SLOW alprazolam dosage form occurred at around 22 hours after dosing, in contrast to the first Cₘₐₓ at 4 hours and a second Cₘₐₓ at 10 hours for the immediate release dosage form.

On Day 6 (FIG. 11B), the SLOW alprazolam dosage form provided less fluctuation in plasma concentration relative to the immediate release formulation. Thus, the SLOW alprazolam dosage form can be further or alternatively characterized as providing a steady-state release rate that results in a plasma concentration fluctuation of 1.0 or less. Plasma concentration fluctuation is a unitless value and is determined by calculating the ratio of the numerical value of the difference between a steady-state Cₘₐₓ (C_{max,ss} ng/mL) and a steady-state minimum plasma drug concentration (C_{min,ss} ng/mL) to the numerical value of an average steady-state plasma drug concentration (C_{ave,ss} ng/mL), the average taken over the relevant time period for which C_{max,ss} and C_{min,ss} are determined. Thus, the plasma concentration fluctuation is a ratio equal to (C_{max,ss}−C_{min,ss})/C_{ave,ss}. The difference in the values of the derived ratios characterize the reduction in the magnitude of peak plasma alprazolam concentrations following continuous (e.g., at least about 3 days) administration of the alprazolam dosage forms compared to peak plasma alprazolam concentrations following administration of conventional immediate-release alprazolam dosage forms. A dosage form configured to provide a release rate that results in a plasma concentration fluctuation of alprazolam that is 1.0 or less is preferred, with dosage forms providing a plasma concentration fluctuation of 0.5 or less being more preferred.

Adverse events resulting from the treatments were reported and tabulated. The data is summarized in Tables 6A-6B.

TABLE 6A
Percent of Subjects Reporting Adverse Events
Following Chronic Alprazolam with > 5% Incidence

| | | Treatment 2¹ | |
|---|---|---|---|
| | Treatment 1¹ | (SLOW | Treatment 3¹ |
| | (IR dosage form) | dosage form) | (placebo) |
| | (n = 36) | (n = 34) | (n = 34) |
| Subjects reporting at | 83.3% (n = 30) | 67.6% (n = 23) | 47.1% (n = 16) |
| least one | | | |
| adverse event | | | |
| Body as a whole | 16.7% (n = 6) | 14.7% (n = 5) | 23.5% (n = 8) |
| Cardiovascular | 13.9% (n = 5) | 8.8% (n = 3) | 5.9% (n = 2) |
| Digestive | 19.4% (n = 7) | 17.6% (n = 6) | 8.8% (n = 3) |
| Nervous | 77.8% (n = 28) | 61.8% (n = 21) | 23.5% (n = 8) |
| Respiratory | 11.1% (n = 4) | 2.9% (n = 1) | 2.9% (n = 1) |
| Skin | 5.6% (n = 2) | 0.0 | 0.0 |
| Special Senses | 5.6% (n = 2) | 0.0 | 0.0 |
| Urogenital | 0.0 | 5.9% (n = 2) | 2.9% (n = 1) |

¹Treatment 1 - immediate release alprazolam (XANAX ®), 1 mg every 3 hours; Treatment 2 - SLOW dosage form (Example 5A), 3 mg once per day; Treatment 3 - placebo

TABLE 6B
Percent of Nervous System Adverse Events
Following Chronic Alprazolam with > 5% Incidence

| | | Treatment 2¹ | |
|---|---|---|---|
| | Treatment 1¹ | (SLOW | Treatment 3¹ |
| | (IR dosage form) | dosage form) | (placebo) |
| | (n = 36) | (n = 34) | (n = 34) |
| Insomnia | 47.2% (n = 17) | 41.2% (n = 14) | 8.8% (n = 3) |
| Somnolence | 36.1% (n = 13) | 14.7% (n = 5) | 2.6% (n = 1) |
| Dizziness | 36.1% (n = 13) | 11.8% (n = 4) | 2.9% (n = 1) |
| Abnormal Dreams | 30.6% (n = 11) | 8.8% (n = 3) | 2.9% (n = 1) |
| Depression | 5.6% (n = 2) | 8.8% (n = 3) | 0.0 |
| Emotional Liability | 5.6% (n = 2) | 2.9% (n = 1) | 2.9% (n = 1) |
| Speech Disorder | 5.6% (n = 2) | 0.0 | 0.0 |

¹Treatment 1 - immediate release alprazolam (XANAX ®), 1 mg every 3 hours; Treatment 2 - SLOW dosage form (Example 5A), 3 mg once per day; Treatment 3 - placebo

Table 6A shows the percent of subjects reporting adverse events following alprazolam treatment, with greater than a 5% incidence. The number of subjects reporting at least one adverse event was lower for subjects treated with the SLOW alprazolam dosage form (23/34) compared to those treated with immediate release alprazolam dosage form (30/36). Most reported adverse events involved the nervous system, and Table 6B provides an inspection of the nervous system adverse events. A lower incidence of somnolence and dizziness was observed when alprazolam was administered from the SLOW dosage form. Thus, patients taking this dosage form are more likely to be able to function normally while initiating therapy.

Accordingly, in another aspect, the invention provides a method to reduce the occurrence of adverse events, and more specifically, nervous system adverse events, by administering a dose of alprazolam in a dosage form the provides a Cₘₐₓ more than 14 hours after administration, more preferably more than 16 hours after administration.

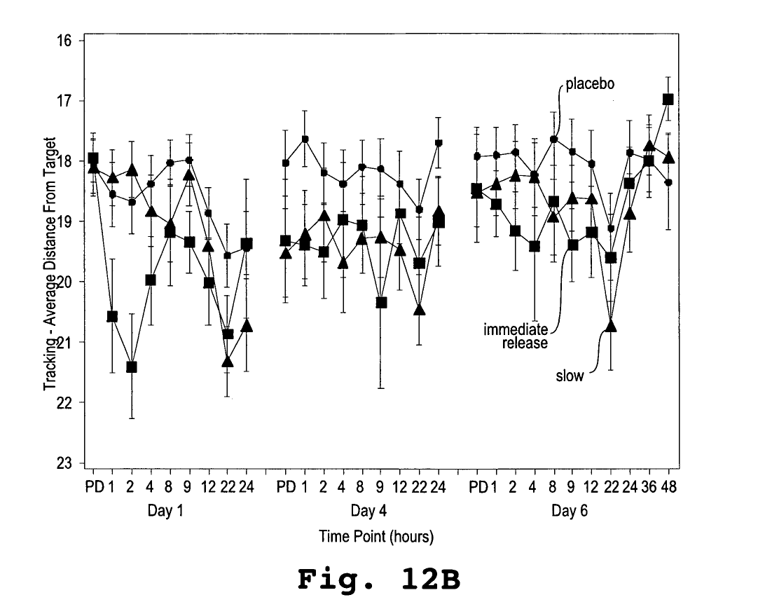

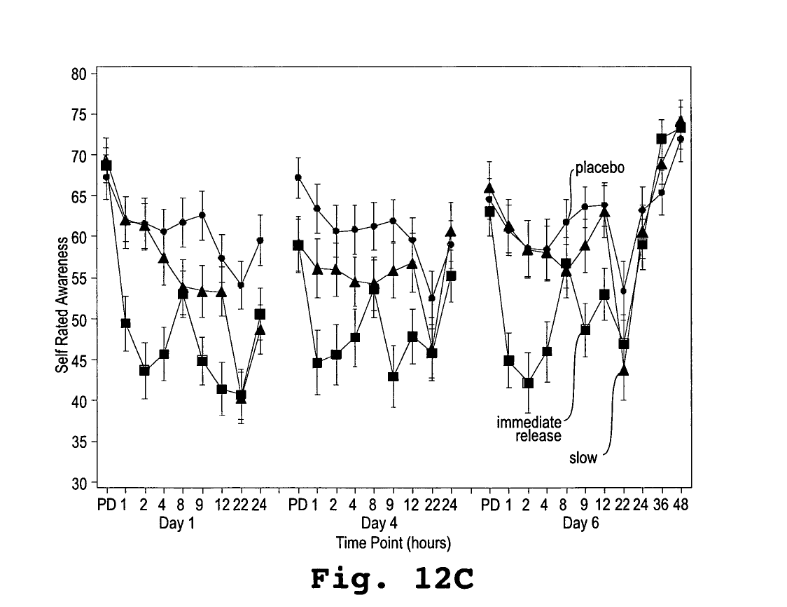

During each arm of the six day treatment period, a selection of tasks from the Cognitive Drug Research's (CDR) computerized cognitive assessment system were administered to the patients, as described in Example 6B. The tests were administered on Days 1, 4, and 6. Results from three of the tests are shown in FIGS. 12A-12C. FIG. 12A shows the data for a Digit Symbol Substitution Test (DSST) assessment for the three treatments. A significant difference is seen on Day 1, where subjects treated with the immediate release dosage form (squares) scored lower than those treated with the SLOW alprazolam dosage form (triangles).

FIG. 12B shows the results for a tracking assessment of average distance from target. The difference in dosage forms is apparent on Day 1 where subjects treated with the immediate release dosage form (squares) scored lower than those treated with the SLOW alprazolam dosage form (triangles).

FIG. 12C shows the results for a self-rated alertness assessment done on days 1, 4, and 6 of treatment. The difference in dosage forms is apparent on Days 1, 4, and 6, where subjects treated with the SLOW alprazolam dosage form (triangles) rated themselves as more alert than did patients treated with the immediate release dosage form (squares).

Several cognitive function tests were also administered to determine whether a lower rate of drug delivery, as provided by the SLOW formulation, translated into less cognitive impairment. The findings suggested faster reaction times; greater accuracy in tasks completed; greater accuracy in tracking a target; and higher alertness with the SLOW formulation, relative to the immediate release formulation.

In summary, the results from this multi-day, multi-dose in vivo study, shows that extent of cognitive impairments caused by alprazolam differed according to the dosage form. The perceived impairments were greater with alprazolam administered as an immediate release formulation than when administered with the SLOW alprazolam dosage form. The difference was most obvious and readily detected on the first day of dosing, however, for a number of measures this advantage provided by the slower rate of delivery was still seen on Day 6, as evidenced by measures of attention, memory, and self-rated alertness.

The data from this in vivo study illustrate that a dosage form providing a lower fluctuation in drug plasma concentration results in a significant reduction in side effects. The relatively slower onset and relatively reduced steady-state plasma concentrations of alprazolam provided by dosage forms according to the present invention reduced sedation, abuse potential, and cognitive impairment. The reduction of such side effects can result in increased patient tolerance as well as enhanced efficacy. It was further shown that the relatively slower onset and relatively reduced steady-state plasma concentrations of alprazolam provided by dosage forms of the present invention also reduced drug liking, which, in turn, reduces the potential for diversion and abuse of alprazolam.

From the foregoing, it can be appreciated that the dosage form described herein is suitable for use in treating conditions responsive to alprazolam. Conditions responsive to alprazolam include, but are not limited to, generalized anxiety disorder, anxiety disorder, panic disorder, anxiety disorder due to general medical condition, panic disorder without agoraphobia, panic disorder with agoraphobia, separation anxiety disorder, adjustment disorder with anxiety, post-traumatic stress disorder, adjustment disorder with mixed anxiety and depressed mood, social anxiety disorder, anxiety attacks, panic attacks, and premenstrual dysphoric disorder. In addition, other disease states and conditions which may or may not manifest in association with central nervous system, but which may be responsive to treatment with alprazolam may also be treated with the dosage forms and methods of the invention. In one embodiment, a method of treating an anxiety disorder is provided, where an alprazolam dosage form is administered for treatment of one or more of the following anxiety disorders: mood disorders, general anxiety disorder, panic disorder, bipolar disorder, social phobias, substance abuse disorders, sleep disorders, stress disorders, and/or conduct disorders.

In conventional therapy for patients with anxiety, alprazolam treatment from an immediate release table is typically initiated with a dose of 0.25 to 0.5 mg three times daily, with dose increments at intervals of 3-4 days to a maximum dose of 4.0 mg per day given in divided doses. Higher doses (up to 10 mg daily) can be used in panic disorders. Therapy with the SLOW dosage forms described herein are provided in unit dosages of between 0.25-25 mg, which are delivered once per day.

### EXAMPLES

Exemplary dosage forms and methods of manufacturing osmotic dosage forms of the present invention are generally described in the examples that follow. All percentages are weight percent unless otherwise noted. The following examples are illustrative of the present invention and should not be considered as limiting the scope of the invention.

### Example 1

### Alprazolam Dosage Form Preparation

A dosage form with a 2 mg dose of alprazolam was manufactured as follows. A binder solution was prepared from poly(vinylpyrrolidone) (Povidone® K29-32, 40 kDa molecular weight) dissolved in water. Poly(ethylene oxide) (Polyox® N-80, 200 kDa molecular weight), sodium chloride (screened with a 20-mesh screen) and poly(vinylpyrrolidone) (Povidone® K29-32, 40 kDa) were added to a Freund Fluid Bed Granulator's bowl. The bowl was attached to the granulator and the granulation process was initiated for effecting granulation. The indicated components as dry powders were air suspended and mixed. Then, the binder solution was sprayed from two nozzles onto the powder. The granulating conditions were monitored during the process as follows: total solution spray rate of 50 mL/min, an exhaust temperature of 21-26° C. and airflow of 200-900 cfm.

While spraying the binder solution, the filter bags were shaken for 10 seconds after every 30-second spray cycle to unglue any possible powder deposits. The granulation process was paused. The desired amount of alprazolam was then added into the granulator bowl. The granulation process was then continued using the same processing conditions. At the end of the solution spraying, the coated granulated particles were continued with the drying process. The machine was turned off, and the coated granules were removed from the granulator. The coated granules were passed through a 7-mesh screen. Next, the dried and screened granulation was transferred to an appropriate container and mixed with butylated hydroxytoluene for 10 minutes. Finally, the granulation was lubricated with of magnesium stearate by mixing for 1 minute.

Next, a push composition was prepared by first making a binder solution from hydroxpropylmethylcellulose (11.2 kDa molecular weight) dissolved in water. Sodium chloride and ferric oxide were sized using a Quadro Comil with a 21-mesh screen. The screened materials, pharmaceutically acceptable poly(ethylene oxide) (Polyox® 303, 7,000 kDa molecular weight) and hydroxpropylmethylcellulose (11.2 kDa molecular weight) were added to a Glatt Fluid Bed Granulator's bowl. The bowl was attached to the granulator and the granulation process was initiated for effecting granulation. The dry powders were air suspended and mixed. Then, the binder solution was sprayed from 3 nozzles onto the powder. The granulating conditions were monitored during the process as follows: total solution spray rate of 700 g/min; inlet temperature 45° C.; and process airflow of 500-4000 m³/hr.

While spraying the binder solution, the filter bags were shaken for 10 seconds every 90 seconds to unglue any possible powder deposits. At the end of the solution spraying, the coated granulated particles were continued with the drying process. The machine was turned off, and the coated granules were removed from the granulator. The coated granules were sized using a Fluid Air mill with a 7-mesh screen. The granulation was transferred to Tote Tumbler, mixed with butylated hydroxytoluene and lubricated with magnesium stearate.

Next, the drug composition and the push composition were compressed into bilayer tablets on the Manesty BB4 Tablet Press. First, the drug composition was added to the die cavity and pre-compressed with a 75-lb force. Then, the push composition was added and the layers were pressed under a pressure head of 1000 lb into 9/32″ (0.714 cm) diameter standard round concave layered arrangements.

The bilayer arrangements were coated with a semi-permeable wall of cellulose acetate (39.8% acetyl content, Eastman Chemical Co. CA398-10) and polyethylene glycol (3350 kDa viscosity-average molecular weight). The wall-forming composition was dissolved in an acetone:water (95:5 wt:wt) cosolvent to make a 5% solids solution. The wall-forming composition was sprayed onto and around the bilayer arrangements in a 24″ Vector HiCoater.

Next, an exit passageway was drilled through the semi-permeable wall to connect the drug layer with the exterior of the dosage system. The residual solvent was removed by drying for a specified time at a specific temperature and relative humidity (e.g., 72 hours at 45° C. and 45% humidity). The osmotic dosage forms were then dried.

The dosage forms prepared by this method were comprised of a 220 mg drug layer containing a 10% overage of alprazolam. The formulation in the drug layer was comprised of

| Component | Weight Percent |
|---|---|
| alprazolam | 1 |
| polyethylene oxide (200 kDa) | 73.5 |
| NaCl | 20 |
| hydroxpropylmethylcellulose | 5 |
| magnesium stearate | 0.5 |

The push layer was 120 mg containing:

| Component | Weight Percent |
|---|---|
| polyethylene oxide (7000 kDa) | 63.6 |
| NaCl | 30.0 |
| hydroxpropylmethylcellulose | 5.0 |
| (HPMC 2910, 5 cps) | |
| iron oxide | 1.0 |
| magnesium stearate | 0.25 |
| butylated hydroxytoluene | 0.08 |

The systems had a 1.83/1.0 drug/push layer ratio.

The semipermeable membrane was 33.2 mg containing cellulose acetate (Eastman Chemical Co. CA398-10)/polyethylene glycol (PEG 3350) in a 97/3 weight ratio was mixed in a 95/5 acetone/water solvent for coating the dosage forms. The systems were dried for 2 days at 50° C. and 50% relative humidity then at 50° C. and ambient relative humidity for 4 hours. A single exit passage having a diameter of 25 mils was drilled on the drug side.

The in vitro dissolution rates of five dosage forms was determined by placing a dosage form in the metal coil sample holders attached to a USP Type VII bath indexer in a constant temperature water bath at 37° C. Aliquots of the release media were injected into a chromatographic system to quantify the amounts of drug released into a medium simulating artificial gastric fluid (AGF) during each testing interval. Five dosage forms were tested in a release medium at 37° C. The dissolution rates are shown in FIGS. 1A-1B.

An average release rate was determined using an iterative calculation to determine the portion of the release profile that was zero order, where values included in the average release rate calculation were within ±5% of the mean release rate. An average release rate is recalculated for each data point, and then that point is checked to verify that it is within ±5% of the recalculated average release rate. This iteration is repeated until the average release rate is determined.

### Example 2

### Alprazolam Dosage Form Performance Comparison in AIF and AGF

Dosage forms comprising 2 mg of alprazolam were prepared as described in Example 1 to have the following specifications.

The drug layer of 210 mg weight contained a 5% overage of alprazolam. The formulation in the drug layer was comprised of:

| Component | Weight Percent |
|---|---|
| alprazolam | 1.0 |
| poly(ethylene oxide) (200 kDa) | 73.5 |
| NaCl | 20.0 |
| hydroxpropylmethylcellulose | 5.0 |
| magnesium stearate | 0.5 |

The push layer had a total weight of 140 mg and was comprised of:

| Component | Weight Percent |
|---|---|
| Poly(ethylene oxide) (7000 kDa) | 63.6 |
| NaCl | 30.0 |
| hydroxpropylmethylcellulose | 5.0 |
| Iron oxide | 1.0 |
| magnesium stearate | 0.25 |
| butylated hydroxytoluene | 0.08 |

The drug composition and the push composition were compressed into bilayer tablets, as described in Example 1, to provide systems with a 1.5/1.0 drug/push layer ratio.

To form the semipermeable membrane, a sufficient amount of cellulose acetate (Eastman Chemical Co. CA398-10) in an acetone/methanol (90/10) solvent mixture was applied to result in a 41.8 mg cellulose acetate coating.

The dosage forms were dried for 2 days at 50° C. and 50% relative humidity then at 50° C. and ambient relative humidity for 15 hours. A single exit port of 25 mils was placed in each dosage form.

The in vitro dissolution rates of the dosage forms were determined by placing a dosage form in the metal coil sample holders attached to a USP Type VII bath indexer in a constant temperature water bath at 37° C. As release media, fluids simulating artificial gastric fluid (AGF, pH 1.2) and artificial intestinal fluid (AIF, pH 6.8) were used. Both artificial release media contained no enzymes. Aliquots of the release media were injected into a chromatographic system to quantify the amounts of drug released during each testing interval. The results are shown in FIGS. 2A-2C.

### Example 3

### In vitro Dissolution Assay for Osmotic Dosage Forms having 0.5 mg and 2 mg Alprazolam

Dosage forms comprising 0.5 mg or 2 mg of alprazolam were prepared as described in Example 1 to have the following specifications.

The drug layer had a total weight of 91 mg weight and was comprised of:

| | Weight Percent | Weight Percent |
|---|---|---|
| | For 0.5 mg Dosage | For 2.0 mg Dosage |
| Component | Form | Form |
| alprazolam | 0.6 | 2.2 |
| poly(ethylene oxide) | 90.03 | 88.53 |
| (200 kDa) | | |
| polyvinylpyrrilodone | 4.0 | 4.0 |
| (Povidone ® K29-32) | | |
| NaCl | 5.0 | 5.0 |
| magnesium stearate | 0.25 | 0.25 |
| iron oxide (green) | 0.10 | 0 |
| butylated hydroxytoluene | 0.02 | 0.02 |

The push layer had a total weight of 75 mg and was comprised of:

| | Weight Percent | Weight Percent |
|---|---|---|
| | For 0.5 mg | For 2.0 mg |
| Component | Dosage Form | Dosage Form |
| poly(ethylene oxide) | 64.3 | 64.3 |
| (7000 kDa) | | |
| NaCl | 30.0 | 30.0 |
| polyvinylpyrrilodone | 5.0 | 5.0 |
| (Povidone ® K29-32) | | |
| ferric oxide (red) | 0.40 | 0.40 |
| magnesium stearate | 0.25 | 0.25 |
| butylated hydroxytoluene | 0.05 | 0.05 |

The drug composition and the push composition were compressed into bilayer tablets, as described in Example 1.

The semipermeable wall of the dosage form was a mixture of 99 wt % cellulose acetate (Eastman Chemical Co. CA398-10) and 1 wt % polyethylene glycol (3350 Da). The mixture was applied to achieve approximately 28 mg of the cellulose acetate/polyethylene glycol on the dosage form.

The dosage forms were dried as described above and two exit ports of 0.634 mm diameter were made in each dosage form. The diameter of each dosage form was 9/32″.

The in vitro dissolution rates of the dosage forms were determined by placing a dosage form in the metal coil sample holders attached to a USP Type VII bath indexer in a constant temperature water bath at 37° C. Aliquots of the release media (water) were injected into a chromatographic system to quantify the amounts of drug released during each testing interval. The results are shown in FIGS. 3A-3C.

### Example 4

### Dosage Forms with Varying Osmagent Content in the Drug Layer

A. Osmotic Dosage Forms with no Osmagent and with 20% NaCl as an Osmagent

Dosage forms with no osmagent in the drug layer or with an osmagent in the drug layer were prepared as described in Example 1 to have the following specifications.

The drug layer had a total weight of 210 mg weight and was comprised of:

| | Weight Percent | Weight Percent |
|---|---|---|
| | For Dosage Form | For Dosage Form |
| Component | without Osmagent | with Osmagent |
| Alprazolam | 1.0 | 1.0 |
| poly(ethylene oxide) | 93.5 | 73.5 |
| (200 kDa) | | |
| hydroxpropylmethylcellulose | 5.0 | 5.0 |
| (HPMC 2910, 5 cps) | | |
| NaCl | 0 | 20.0 |
| magnesium stearate | 0.50 | 0.50 |

The push layer had a total weight of 140 mg and was comprised of:

| | Weight Percent | Weight Percent |
|---|---|---|
| | For Dosage Form | For Dosage Form |
| Component | without Osmagent | with Osmagent |
| poly(ethylene oxide) | 63.67 | 63.67 |
| (7000 kDa) | | |
| NaCl | 30.0 | 30.0 |
| hydroxpropylmethylcellulose | 5.0 | 5.0 |
| (HPMC 2910, 5 cps) | | |
| ferric oxide (red) | 1.0 | 1.0 |
| magnesium stearate | 0.25 | 0.25 |
| butylated hydroxytoluene | 0.08 | 0.08 |

The drug composition and the push composition were compressed into bilayer tablets, as described in Example 1.

To form the semipermeable wall of the dosage forms, a total amount of 45 mg of cellulose acetate (Eastman Chemical Co. CA398-10) was applied to the dosage forms having no osmagent and a total amount of 46 mg was applied to the dosage forms having 20% sodium chloride as osmagent.

The dosage forms were dried as described above and a single exit port of 0.559 mm diameter were made in each dosage form. The diameter of each dosage form was ⅜″.

The systems were tested for release of drug in a USP Type VII bath indexer. Aliquots of the release media were injected into a chromatographic system to quantify the amounts of drug released during each testing interval. The results are shown in FIGS. 5A-5C.

B. Osmotic Dosage Forms with 20% NaCl or 30% NaCl as an Osmagent

Dosage forms were prepared as described in Example 1 to have the following specifications.

The drug layer had a total weight of 210 mg weight and was comprised of:

| | Weight Percent | Weight Percent |
|---|---|---|
| | For Dosage Form | For Dosage Form |
| Component | without Osmagent | with Osmagent |
| Alprazolam | 1.0 | 1.0 |
| poly(ethylene oxide) | 73.5 | 63.5 |
| (200 kDa) | | |
| hydroxpropylmethylcellulose | 5.0 | 5.0 |
| (HPMC 2910, 5 cps) | | |
| NaCl | 20.0 | 30.0 |
| magnesium stearate | 0.50 | 0.50 |

The push layer had a total weight of 140 mg and was comprised of:

| | Weight Percent | Weight Percent |
|---|---|---|
| | For Dosage Form | For Dosage Form |
| Component | without Osmagent | with Osmagent |
| poly(ethylene oxide) | 63.67 | 63.67 |
| (7000 kDa) | | |
| NaCl | 30.0 | 30.0 |
| hydroxpropylmethylcellulose | 5.0 | 5.0 |
| (HPMC 2910, 5 cps) | | |
| Ferric oxide (red) | 1.0 | 1.0 |
| magnesium stearate | 0.25 | 0.25 |
| butylated hydroxytoluene | 0.08 | 0.08 |

The drug composition and the push composition were compressed into bilayer tablets, as described in Example 1.

To form the semipermeable wall of the dosage forms, a total amount of 42 mg of cellulose acetate (Eastman Chemical Co. CA398-10) was applied to the dosage forms having 20% sodium chloride as osmagent and a total amount of 41 mg was applied to the dosage forms having 30% sodium chloride as osmagent.

The dosage forms were dried as described above and a single exit port of 0.559 mm diameter were made in each dosage form. The diameter of each dosage form was ⅜″.

The systems were tested for release of drug in a USP Type VII bath indexer. Aliquots of the release media were injected into a chromatographic system to quantify the amounts of drug released during each testing interval. The results are shown in FIGS. 6A-6C.

### Example 5

### In vivo Evaluation of Alprazolam Dosage Forms

A. Dosage Form Compositions

Two dosage forms referred to as a SLOW dosage form and a FAST dosage form, both having 1.0 mg of alprazolam, were prepared as described in Example 1 to have the following specifications.

The drug layer had a total weight of 91 mg weight and was comprised of:

| | Weight Percent | Weight Percent |
|---|---|---|
| Component | For SLOW system | For FAST system |
| Alprazolam | 1.10 | 1.10 |
| poly(ethylene oxide) | 74.63 | 74.63 |
| (200 kDa) | | |
| polyvinylpyrrilodone | 4.0 | 4.0 |
| (Polyox ® K29-32) | | |
| NaCl | 20.0 | 20.0 |
| magnesium stearate | 0.25 | 0.25 |
| butylated hydroxytoluene | 0.02 | 0.02 |

The push layer had a total weight of 61 mg and was comprised of:

| | Weight Percent | Weight Percent |
|---|---|---|
| Component | For SLOW system | For FAST system |
| poly(ethylene oxide) | 63.6 | 63.6 |
| (7000 kDa) | | |
| NaCl | 30.0 | 30.0 |
| hydroxpropylmethylcellulose | 5.0 | 5.0 |
| (HPMC 2910, 5 cps) | | |
| Ferric oxide (red) | 0.40 | 0.40 |
| stearic acid | 0.95 | 0.95 |
| butylated hydroxytoluene | 0.05 | 0.05 |

The drug composition and the push composition were compressed into bilayer tablets, as described in Example 1.

The SLOW and FAST dosage forms differed only in the thickness of the semipermeable wall. For both formulations, the wall was comprised of a mixture of 99 wt % cellulose acetate (Eastman Chemical Co. CA398-10) and 1 wt % polyethylene glycol (3350 Da). A total of 26 mg of the mixture in acetone was applied to the SLOW dosage forms. A total of 16 mg of the mixture in acetone was applied to the FAST dosage forms.

The dosage forms were dried as described above and a single exit port of 0.65 mm diameter was made in each dosage form. The diameter of each dosage form was 9/32″.

In in vitro dissolution assays, the SLOW dosage form had a T₉₀ of 20 hours and the FAST dosage form had a T₉₀ of 10 hours.

B. In vivo Study

Twenty-four adults (18 to 55 years) were enrolled for a single-center, single-dose, placebo-controlled, double-blind, six-treatment, five-period, randomized, incomplete block, crossover study. The adult subjects had a history of sedative or tranquilizer drug use. The subjects (i) were experienced users of two or more central nervous system (CNS) depressants such as benzodiazepines, barbiturates, non-benzodiazepine sedatives, and hypnotics including cannabis and alcohol (<60 g/day) in the past year, with at least one in tablet or capsule form and (ii) had a positive response to a secobarbital screening test.

Each subject received five of six treatments, identified as Treatments A-F:

| | |
|---|---|
| Treatment A | placebo (SLOW¹ dosage form with no |
| | alprazolam) |
| Treatment B | SLOW¹ alprazolam; 1 mg |
| Treatment C | SLOW¹ alprazolam; 2 × 1 mg |
| Treatment D | SLOW¹ alprazolam; 3 × 1 mg |
| Treatment E | FAST² alprazolam; 2 × 1 mg |
| Treatment F | immediate-release alprazolam³; 2 mg |

¹SLOW dosage form composition provided above in part A.
²FAST dosage form composition provided above in part A.
³immediate-release alprazolam available under the tradename XANAX ®.

Treatments were separated by a washout period of not less than 4 days and not more than 21 days.

Plasma samples were collected at 0 (predose), 0.5, 1, 2, 5, 8, 13, 21, 24, and 30 hours after oral dosing for measurement of alprazolam concentrations. The pharmacokinetic results are shown in Table 1 and in FIG. 7.

The pharmacodynamic effects of the treatment regimens were evaluated using various assessments of the potential for abuse liability including:
(1) Cole/Addiction Research Center Inventory (ARCI) Abuse Potential score;
(2) Cole/ARCI stimulation—euphoria scale;
(3) Cole/ARCI stimulation—abuse potential scale;
(4) Monetary Value of Drug;
(5) Drug Effects Questionnaire (DEQ).
   Results for (4) and (5) are shown in FIG. 8 and in Table 2, respectively.

To evaluate the potential for psychomotor impairment, additional tests were conducted including:
(6) Digit Symbol Substitution Test (DSST); and
(7) Manual Tracking Test (% over road)

Results are shown in FIG. 9 and in Tables 3A-3B.

TABLE 3A
Summary of Psychomotor Assessments: Manual Tracking Test
(Data Reported are p-value (estimate of difference))

| | Trmt B- | Trmt C- | Trmt D- | Trmt E- | Trmt F- |
|---|---|---|---|---|---|
| | Trmt A | Trmt A | Trmt A | Trmt A | Trmt A |
| Mean maximum | 0.103 | 0.002 | <0.001 | <0.001 | <0.001 |
| distance (mm) | (9.11) | (17.01) | (24.62) | (22.78) | (50.93) |
| Mean percent over | 0.010 | 0.023 | <0.001 | <0.001 | <0.001 |
| road | (−3.67) | (−3.20) | (−5.45) | (−4.92) | (−14.11) |
| Mean RMS | 0.065 | 0.040 | <0.001 | 0.001 | <0.001 |
| distance (mm) | (3.59) | (3.91) | (7.28) | (6.23) | (19.39) |

Treatment A = placebo; Treatment B = 1 mg SLOW; Treatment C = 2 × 1 mg SLOW; Treatment D = 3 × 1 mg SLOW; Treatment E = 2 × 1 mg FAST; Treatment F = 2 mg immediate release

TABLE 3B
Summary of Psychomotor Assessments: Digital Symbol
Substitution Test (Data Reported are p-value
(estimate of difference))

| | Trmt B − Trmt A | Trmt C − Trmt A | Trmt D − Trmt A | Trmt E − Trmt A | Trmt F − Trmt A |
|---|---|---|---|---|---|
| Symbols | 0.612 | 0.800 | 0.147 | 0.063 | <0.001 |
| completed | (−0.23) | (−0.11) | (−0.65) | (−0.81) | (−3.33) |
| Symbols correct | 0.458 | 0.182 | 0.937 | 0.889 | <0.001 |
| | (0.37) | (0.65) | (0.04) | (−0.07) | (−2.76) |
| Mean time to start | 0.262 | 0.259 | 0.899 | 0.755 | <0.001 |
| (correct, msec) | (−32.20) | (−31.86) | (3.65) | (−8.65) | (122.10) |
| Mean time to finish | 0.589 | 0.692 | 0.406 | 0.359 | <0.001 |
| (correct, msec) | (72.25) | (51.94) | (111.11) | (118.05) | (1141.57) |

Treatment A = Placebo; Treatment B = 1 mg SLOW; Treatment C = 2 × 1 mg SLOW; Treatment D = 3 × 1 mg SLOW; Treatment E = 2 × 1 mg FAST; Treatment F = 2 mg immediate release

To evaluate the potential for sedation, an additional complement of tests were conducted including:
(8) Tufts University Benzodiazepine Scale (TUBS);
(9) Addiction Research Center Inventory (ARCI) pentobarbital, chlorpromazine, alcohol group (PCAG) scale;
(10) Bond and Lader mental sedation score;
(11) Bond and Lader Physical sedation score;
(12) Cole/ARCI Sedation—Mental; and
(13) Cole/ARCI Sedation—Motor

Results are shown in Table 4 and in FIGS. 10A-10C.

TABLE 4
Summary of Pharmacodynamic Parameters Assessing Sedation
(Data Reported are p-value (estimate of difference))

| | | | | | |
|---|---|---|---|---|---|
| Test | Trmt B¹ − Trmt A | Trmt C − Trmt A | Trmt D − Trmt A | Trmt E − Trmt A | Trmt F − Trmt A |

Tufts University Benzodiazepine Scale (TUBS)

| | | | | | |
|---|---|---|---|---|---|
| Sedation item | 0.385 | 0.808 | 0.010 | 0.023 | <0.001 |
| mean | (2.83) | (0.78) | (8.38) | (7.15) | (19.79) |

Cole/ARCI

| | | | | | |
|---|---|---|---|---|---|
| Sedation-Motor | 0.086 | 0.766 | <0.001 | 0.002 | <0.001 |
| | (1.00) | (0.17) | (2.38) | (1.72) | (3.78) |
| Sedation-Mental | 0.265 | 0.336 | 0.001 | 0.034 | <0.001 |
| | (1.05) | (0.89) | (3.28) | (1.94) | (6.19) |
| PCAG² | 0.052 | 0.333 | <0.001 | 0.026 | <0.001 |
| | (1.97) | (0.96) | (3.62) | (2.17) | (5.96) |

Bond and Lader

| | | | | | |
|---|---|---|---|---|---|
| Mental Sedation | 0.021 | 0.706 | 0.450 | 0.980 | 0.206 |
| | (−9.22) | (1.48) | (3.02) | (0.10) | (4.95) |
| Physical Sedation | 0.152 | 0.625 | 0.335 | 0.217 | 0.992 |
| | (−5.66) | (−1.89) | (−3.80) | (−4.70) | (0.04) |

¹Treatment A = Placebo; Treatment B = 1 mg SLOW; Treatment C = 2 × 1 mg SLOW; Treatment D = 3 × 1 mg SLOW; Treatment E = 2 mg FAST; Treatment F = 2 mg immediate release
²PCAG = Pentobarbitol, chlorpromazine, alcohol group scale values reported are p-values (estimates of difference in contrast)

### Example 6

### Multi-Dosing In vivo Study

A. Dosage Form Compositions

Dosage forms comprising 1 mg alprazolam, identified as SLOW alprazolam dosage forms, were prepared as described in Example 5A. Placebo dosage forms were identical in composition, except contained no alprazolam.

B. In vivo Study

Thirty-six adults were enrolled for a randomized, placebo-controlled, multiple-dose, crossover study. Each subject received three treatments, Treatment 1, Treatment 2, Treatment 3, with a minimum washout period of 7 days between treatments. The treatments were:

| | |
|---|---|
| Treatment 1 | immediate release alprazolam, |
| (comparative control) | 1 mg tablet orally every 8 hours |
| | for 6 days |
| Treatment 2 | SLOW¹ alprazolam; 3 × 1 mg orally |
| | once per day for 6 days |
| Treatment 3 | SLOW¹ alprazolam placebo, 3 |
| (placebo) | systems orally once per day for 6 |
| | days |

¹immediate-release alprazolam available under the tradename XANAX ®.
²SLOW dosage form composition provided above in Example 5A.

Plasma samples were collected at 0 (predose) and at regular intervals over the six day testing period for measurement of alprazolam concentrations. The plasma concentrations on Day 1 and on Day 6 are shown in FIGS. 11A-11B, respectively.

A selection of tasks from the Cognitive Drug Research's (CDR) computerized cognitive assessment system were administered, parallel forms of the tests being presented on each testing session. On Days 1 and 4 CDR testing took place at 0 (pre-dose), 1, 2, 4, 8, 9, 12, 22 and 24 hours post-dose. On Day 6 CDR testing took place at 0 (pre-dose), 1, 2, 4, 8, 9, 12, 22, 24, 36 and 48 hours post-dose. The tests were administered in the following order: Immediate Word Recall; Simple Reaction Time; Digit Vigilance; Choice Reaction Time; Tracking; Digit Symbol Substitution Test (DSST); and Delayed Word Recall. Results for the DSST are shown in FIG. 12A and for the tracking in FIG. 12B.

In addition, subjects were administered the Bond-Lader VAS of Mood and Alertness (Bond and Lader, 1974). These tests were administered on Days 1, 4, and 6. Results for the self-rated alertness are shown in FIG. 12C.

1. A dosage form comprising a dose of alprazolam, said dosage form effective to provide a dissolution rate where between 25% and 60% of the dose is released 10 hours after exposure to an aqueous environment.

2. The dosage form of claim 1 effective to provide a dissolution rate where between 35% and 55% of the dose is released 10 hours after exposure to an aqueous environment.

3. The dosage form of claim 1 effective to provide a dissolution rate where less than 20% of the dose is released 2 hours after exposure to an aqueous environment.

4. A dosage form comprising a dose of alprazolam, said dosage form effective to provide a dissolution rate where between 30% and 80% of the dose is released 12 hours after exposure to an aqueous environment.

5. The dosage form of claim 4 effective to provide a dissolution rate where between 40% and 70% of the dose is released 10 hours after exposure to an aqueous environment.

6. The dosage form of claim 4 effective to provide a dissolution rate where less than 20% of the dose is released 2 hours after exposure to an aqueous environment

7. The dosage form of claim 1, wherein the dosage form is an osmotic dosage form.

8. The dosage form of claim 7 comprised of (i) a push layer; (ii) drug layer comprising alprazolam; (iii) a semipermeable wall provided around the push layer and the drug layer; and (iv) an exit.

9. The dosage form of claim 7 comprised of (i) a semipermeable wall provided around an osmotic formulation comprising an alprazolam formulation, an osmagent, and an osmopolymer; and (ii) an exit.

10. The dosage form of claim 4, wherein the dosage form is an osmotic dosage form.

11. The dosage form of claim 10 comprised of (i) a push layer; (ii) drug layer comprising alprazolam; (iii) a semipermeable wall provided around the push layer and the drug layer; and (iv) an exit.

12. The dosage form of claim 10 comprised of (i) a semipermeable wall provided around an osmotic formulation comprising an alprazolam formulation, an osmagent, and an osmopolymer; and (ii) an exit.

13. The dosage form of claim 1, wherein the dosage form provides a total daily dose of between 0.25-25 mg.

14. The dosage form of claim 13, where in the dosage form provides a total daily dose of between 0.5 and 6 mg.

15. A dosage form, comprising, a dose of alprazolam, said dosage form effective to provide an in vitro release profile where
(i) less than 20% of the dose is released 2 hours after exposure to an aqueous environment;
(ii) between 25% and 65% of the dose is released 10 hours after exposure to an aqueous environment; and
(iii) greater than 85% of the dose is released 24 hours after exposure to an aqueous environment.

16. A dosage form for delivery of alprazolam, comprising a dose of alprazolam, the dosage form being configured to release at least about 10% of the dose 16 hours after exposure to an aqueous environment.

17. The dosage form of claim 16, wherein at least about 15% of the dose is released 16 hours after exposure to an aqueous environment.

18. A dosage form for delivery of alprazolam, comprising a dose of alprazolam, the dosage form being configured to release at least about 15% of the dose 14 hours after exposure to an aqueous environment.

19. The dosage form of claim 18, wherein at least about 20% of the dose is released 14 hours after exposure to an aqueous environment.

20. A dosage form for delivery of alprazolam, comprising a dose of alprazolam, the dosage form being configured to release at least about 25% of the dose 12 hours after exposure to an aqueous environment.

21. The dosage form of claim 20, wherein at least about 30% of the dose is released 12 hours after exposure to an aqueous environment.

22. The dosage form of claim 16, wherein the dosage form is an osmotic dosage form.

23. The dosage form of claim 18, wherein the dosage form is an osmotic dosage form.

24. The dosage form of claim 20, wherein the dosage form is an osmotic dosage form.

25. The dosage form of claim 22 comprised of (i) a push layer; (ii) drug layer comprising alprazolam; (iii) a semipermeable wall provided around the push layer and the drug layer; and (iv) an exit.

26. The dosage form of claim 22 comprised of (i) a semipermeable wall provided around an osmotic formulation comprising an alprazolam formulation, an osmagent, and an osmopolymer; and (ii) an exit.

27. The dosage form of claim 23 comprised of (i) a push layer; (ii) drug layer comprising alprazolam; (iii) a semipermeable wall provided around the push layer and the drug layer; and (iv) an exit.

28. The dosage form of claim 23 comprised of (i) a semipermeable wall provided around an osmotic formulation comprising an alprazolam formulation, an osmagent, and an osmopolymer; and (ii) an exit.

29. The dosage form of claim 24 comprised of (i) a push layer; (ii) drug layer comprising alprazolam; (iii) a semipermeable wall provided around the push layer and the drug layer; and (iv) an exit.

30. The dosage form of claim 24 comprised of (i) a semipermeable wall provided around an osmotic formulation comprising an alprazolam formulation, an osmagent, and an osmopolymer; and (ii) an exit.

31. The dosage form of claim 16, wherein the dosage form provides a total daily dose of between 0.25-25 mg.

32. The dosage form of claim 18, wherein the dosage form provides a total daily dose of between 0.25-25 mg.

33. The dosage form of claim 20, wherein the dosage form provides a total daily dose of between 0.25-25 mg.

34. A dosage form for delivering alprazolam comprising a drug formulation including a desired dose of alprazolam, wherein the dosage form is configured to release alprazolam at a rate that ranges between 2% of the total dose of alprazolam per hour to 7% of the total dose of alprazolam per hour over the period of between 2 and 20 hours after exposure to an aqueous environment.

35. The dosage form of claim 34 wherein the dosage form is configure to release alprazolam at a rate that ranges between 2% of the total dose of alprazolam per hour to 7% of the total dose of alprazolam per hour over the period of between 2 and 16 hours after exposure to an aqueous environment.

36. The dosage form of claim 34 wherein the dosage form is configure to release alprazolam at a rate that ranges between 2% of the total dose of alprazolam per hour to 7% of the total dose of alprazolam per hour over the period of between 2 and 12 hours after exposure to an aqueous environment.

37. A dosage form comprising a dose of alprazolam, said dosage form effective to provide cumulative amount of drug released in vivo of between 25% and 60% of the total dose 10 hours after oral delivery.

38. The dosage form of claim 37 effective to release between 35% and 55% of the dose 10 hours after oral delivery.

39. The dosage form of claim 37 effective to release less than 20% of the dose 2 hours after oral delivery.

40. A dosage form comprising a dose of alprazolam, said dosage form effective to release between 30% and 80% of the dose 12 hours after oral delivery.

41. The dosage form of claim 40 effective to release between 40% and 70% of the dose 10 hours after oral delivery.

42. The dosage form of claim 40 effective to release less than 20% of the dose 2 hours after oral delivery.

43. The dosage form of claim 37, wherein the dosage form is an osmotic dosage form.

44. The dosage form of claim 43 comprised of (i) a push layer; (ii) drug layer comprising alprazolam; (iii) a semipermeable wall provided around the push layer and the drug layer; and (iv) an exit.

45. The dosage form of claim 43 comprised of (i) a semipermeable wall provided around an osmotic formulation comprising an alprazolam formulation, an osmagent, and an osmopolymer; and (ii) an exit.

46. The dosage form of claim 40, wherein the dosage form is an osmotic dosage form.

47. The dosage form of claim 46 comprised of (i) a push layer; (ii) drug layer comprising alprazolam; (iii) a semipermeable wall provided around the push layer and the drug layer; and (iv) an exit.

48. The dosage form of claim 46 comprised of (i) a semipermeable wall provided around an osmotic formulation comprising an alprazolam formulation, an osmagent, and an osmopolymer; and (ii) an exit.

49. The dosage form of claim 37, wherein the dosage form provides a total daily dose of between 0.25-25 mg.

50. The dosage form of claim 49, where in the dosage form provides a total daily dose of between 0.5 and 6 mg.

51. A dosage form, comprising alprazolam, wherein the dosage form provides a maximum attained alprazolam plasma concentration (Cₘₐₓ) more than 14 hours after administration.

52. The dosage form of claim 51, wherein the Cmax occurs more than 16 hours after administration.

53. A dosage form comprised of a desired dose of alprazolam, said dosage form being effective to provide a dose normalized ratio of maximum attained alprazolam plasma concentration (Cmax) to time to reach maximum attained alprazolam plasma concentration (Tmax) of less than 0.5.

54. The dosage form of claim 51, wherein the dosage form provides a total daily dose of between 0.25-25 mg.

55. A dosage form comprising alprazolam, said dosage form providing a dose-normalized area under the curve of less than about 110 ng·hr/mL·mg.

56. The dosage form of claim 55, wherein said dose-normalized area under the curve is greater than 70 ng·hr/mL·mg.

57. The dosage form of claim 55, wherein said dosage form is an osmotic dosage form.

58. The dosage form of claim 57, comprised of (i) a push layer; (ii) drug layer comprising alprazolam; (iii) a semipermeable wall provided around the push layer and the drug layer; and (iv) an exit.

59. The dosage form of claim 57, comprised of (i) a semipermeable wall provided around an osmotic formulation comprising an alprazolam formulation, an osmagent, and an osmopolymer; and (ii) an exit.

60. A method for administering alprazolam to a human subject, comprising administering a dosage form effective to provide an in vitro release profile where between 25% and 60% of the dose is released 10 hours after exposure to an aqueous environment.

61. A method for administering alprazolam to a human subject, comprising administering a dosage form effective to provide a ratio of maximum attained alprazolam plasma concentration (Cmax) more than 14 hours after administration.

62. A method for administering alprazolam to a human subject, comprising administering a dosage form effective to provide a dose-normalized area under the curve of less than about 110 ng·hr/mL·mg.

63. A method of treating a condition responsive to alprazolam, comprising administering a dosage form effective to provide a dose-normalized area under the curve of less than about 110 ng·hr/mL·mg.

64. The method according to claim 63, wherein said administering comprises a once daily administration of a dose between 0.25-25 mg.

65. The method of claim 63, wherein said administering comprises administering an osmotic dosage form.

66. The method of claim 65, wherein said dosage form is comprised of (i) a push layer; (ii) drug layer comprising alprazolam; (iii) a semipermeable wall provided around the push layer and the drug layer; and (iv) an exit.

67. The method of claim 65, wherein said dosage form is comprised of (i) a semipermeable wall provided around an osmotic formulation comprising an alprazolam formulation, an osmagent, and an osmopolymer; and (ii) an exit.

68. The method of claim 63, wherein the condition responsive to alprazolam is an anxiety disorder.

69. The method of claim 68, wherein said anxiety disorder is selected from the group consisting of mood disorders, general anxiety disorder, panic disorder, bipolar disorder, social phobias, substance abuse disorders, sleep disorders, stress disorders, conduct disorders.
